(19) United States
(12) Patent Application Publication
(10) Pub. No.: US 2023/0364132 A1
(43) Pub. Date: Nov. 16, 2023

(54) GOLD CLUSTERS, COMPOSITIONS, AND METHODS FOR TREATMENT OF CEREBRAL STROKES

(71) Applicant: SHENZHEN PROFOUND VIEW PHARMACEUTICAL TECHNOLOGY CO., LTD., SHENZHEN (CN)

(72) Inventor: Taolei SUN, Wuhan (CN)

(73) Assignee: SHENZHEN PROFOUND VIEW PHARMACEUTICAL TECHNOLOGY CO., LTD., SHENZHEN, GD (CN)

(21) Appl. No.: 18/249,530

(22) PCT Filed: Aug. 16, 2021
(86) PCT No.: PCT/CN2021/112729

§ 371 Date: Apr. 18, 2023

Related U.S. Application Data
(63) Continuation-in-part of application No. PCT/CN2020/132280, filed on Nov. 27, 2020.

(51) Int. Cl. A61K 33/242; A61K 47/62; A61P 9/04; A61K 9/51

(52) U.S. Cl. CPC A61K 33/242; A61K 47/62; A61P 9/04; A61K 9/51

(57) ABSTRACT

Ligand-bound gold clusters and compositions comprising the ligand-bound gold clusters are used for treating cerebral stroke and manufacturing a medicament for treatment of cerebral stroke. Methods for treating cerebral stroke.

### FIELD OF THE INVENTION

The present invention relates to the technical field of brain illness treatment, particularly to ligand-bound gold clusters (AuCs), composition comprising the ligand-bound AuCs, use of the ligand-bound AuCs to prepare medications for treatment of cerebral strokes, and methods employing the ligand-bound AuCs and composition for treatment of cerebral strokes.

### BACKGROUND OF THE INVENTION

A stroke occurs when a blood vessel is either blocked by a clot or encountered ruptures. There are three types of strokes, i.e. cerebral hemorrhagic stroke, cerebral ischemic stroke, and transient ischemic attack (TIA).

Cerebral hemorrhagic stroke is caused by a blood vessel rupturing and preventing blood flow to the brain. The common symptoms include sudden weakness, paralysis in any part of the body, inability to speak, vomiting, difficulty walking, coma, loss of consciousness, stiff neck and dizziness. No specific medication is available.

Cerebral ischemic stroke, also known as brain ischemia and cerebral ischemia, represents one of the most prevalent pathologies in humans and is a leading cause of death and disability. Cerebral ischemic stroke is accounting for approximately 87 percent of all strokes. Cerebral ischemic stroke is caused by a blockage such as a blood clot or plaque in an artery that supplies blood to the brain, where the blockage appears at the neck or in the skull, and reduces the blood flow and oxygen to the brain, leading to damage or death of brain cells. If blood circulation is not restored quickly, brain damage can be permanent.

Specific symptoms of a cerebral ischemic stroke depend on what region of the brain is affected. Common symptoms for most ischemic stroke include vision problems, weakness or paralysis in limbs, dizziness and vertigo, confusion, loss of coordination, and drooping of face on one side. Once symptoms start, it is crucial to get treatment as quickly as possible, making it less likely that damage becomes permanent.

The available treatment for cerebral ischemic stroke is very limited. The main clinically available treatment drug for cerebral ischemic stroke is tissue plasminogen activator (tPA) that breaks up clots, but the tPA has to be given intravenously within four and a half hours from the start of a stroke to be effective. However, tPA causes bleeding so that patients cannot be treated with tPA if they have a history of hemorrhagic stroke, bleeding in the brain, and recent major surgery or head injury. Long-term treatments include aspirin or an anticoagulant to prevent further clots.

Amani et al. disclose that OX26@GNPs formed by conjugating of OX26-PEG to the surface of 25 nm colloidal gold nanoparticles significantly increased the infarcted brain tissue, and bare GNPs and PEGylated GNPs had no effect on the infarct volume; their results showed that OX26 @ GNPs are not suitable for treatment of cerebral strokes.

Zheng et al. disclose that in their OGD/R injury rat model, 20 nm Au-NPs increased cell viability, alleviated neuronal apoptosis and oxidative stress, and improved mitochondrial respiration. However, Zheng et al. also demonstrated that 5 nm Au NPs showed opposite effects, not suitable for treatment of cerebral strokes.

TIA is caused by a temporary clot. The common symptoms include weakness, numbness or paralysis on one side of the body, slurred or garbled speech, blindness, and vertigo. No specific medication is available.

The treatment of cerebral strokes must be timely to avoid or alleviate a patient's neurological dysfunction or death resulted from the injury or death of brain nerve cells caused by cerebral ischemia or cerebral hemorrhage. There are no specific therapeutic drugs for cerebral hemorrhagic stroke and TIA; the main drug such as tPA for treatment of cerebral ischemic stroke causes bleeding; thus, only after a patient has been affirmatively diagnosed with cerebral ischemic stroke but not cerebral hemorrhage stroke, will a decision be made to use drugs for the treatment of cerebral ischemic stroke. However, this process of affirmative diagnosis costs the precious time for timely treating cerebral ischemic stroke.

There remains a need for effective method and medications for treatment of cerebral strokes, especially for the drugs that can have therapeutic effects on both cerebral hemorrhage stroke and cerebral ischemic stroke. Since the causes for cerebral hemorrhage stroke and cerebral ischemic stroke are fundamentally different, all drug developments in the literature are directed to either cerebral hemorrhage stroke or cerebral ischemic stroke; thus, it has been an unimaginable challenge or could be characterized as a taboo in the industry to develop a drug that can treat patients with cerebral strokes, whether the patient is with cerebral hemorrhage stroke or cerebral ischemic stroke.

### SUMMARY OF THE INVENTION

The present invention provides a ligand-bound gold clusters for use of treatment of cerebral stroke in a subject, a method of treating the cerebral stroke in a subject with a ligand-bound gold clusters, and a ligand-bound gold clusters for use of manufacture of medicament for treatment of the cerebral stroke in a subject.

In certain embodiments, a ligand-bound gold cluster is used for treatment of cerebral stroke in a subject, wherein the ligand-bound gold cluster comprises a gold core, and a ligand bound to the gold core; wherein the cerebral stroke is selected from the group consisting of cerebral hemorrhagic stroke, cerebral ischemic stroke, and transient ischemic attack (TIA).

In certain embodiments of the ligand-bound gold cluster for use of treatment of cerebral stroke, the gold core has a diameter in the range of 0.5-3 nm.

In certain embodiments of the ligand-bound gold cluster for use of treatment of cerebral stroke, the gold core has a diameter in the range of 0.5-2.6 nm.

In certain embodiments of the ligand-bound gold cluster for use of treatment of cerebral stroke, the ligand is one selected from the group consisting of L-cysteine and its derivatives, D-cysteine and its derivatives, cysteine-containing oligopeptides and their derivatives, and other thiol-containing compounds.

In certain embodiments of the ligand-bound gold cluster for use of treatment of cerebral stroke, the L-cysteine and its derivatives are selected from the group consisting of L-cysteine, N-isobutyryl-L-cysteine (L-NIBC), and N-acetyl-L-cysteine (L-NAC), and wherein the D-cysteine and its derivatives are selected from the group consisting of D-cysteine, N-isobutyryl-D-cysteine (D-NIBC), and N-acetyl-D-cysteine (D-NAC).

In certain embodiments of the ligand-bound gold cluster for use of treatment of cerebral stroke, the cysteine-containing oligopeptides and their derivatives are cysteine-containing dipeptides, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

In certain embodiments of the ligand-bound gold cluster for use of treatment of cerebral stroke, the cysteine-containing oligopeptides and their derivatives are cysteine-containing tripeptides, wherein the cysteine-containing tripeptides are selected from the group consisting of glycine-L(D)-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

In certain embodiments of the ligand-bound gold cluster for use of treatment of cerebral stroke, the cysteine-containing oligopeptides and their derivatives are cysteine-containing tetrapeptides, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

In certain embodiments of the ligand-bound gold cluster for use of treatment of cerebral stroke, the cysteine-containing oligopeptides and their derivatives are cysteine-containing pentapeptide, wherein the cysteine-containing pentapeptides are selected from the group consisting of Cysteine-Aspartic acid-Glutamic acid-Valine-Aspartic acid (CDEVD) and Aspartic acid-Glutamic acid-Valine-Aspartic acid-Cysteine (DEVDC).

In certain embodiments of the ligand-bound gold cluster for use of treatment of cerebral stroke, the other thiol-containing compounds are selected from the group consisting of 1-[(2 S)-2-methyl-3-thiol-1-oxopropyl]-L(D)-proline, thioglycollic acid, mercaptoethanol, thiophenol, D-3-trolovol, N-(2-mercaptopropionyl)-glycine, dodecyl mercaptan, 2-aminoethanethiol (CSH), 3-mercaptopropionic acid (MPA), and 4-mercaptobenoic acid (p-MBA).

In certain embodiments, a ligand-bound gold cluster (AuC) is used for manufacture of a medicament for the treatment of cerebral stroke in a subject, wherein the ligand-bound gold cluster comprises a gold core, and a ligand bound to the gold core; wherein the cerebral stroke is selected from the group consisting of cerebral hemorrhagic stroke, cerebral ischemic stroke, and transient ischemic attack (TIA).

In certain embodiments of the ligand-bound gold cluster (AuC) for use of manufacture of a medicament, the gold core has a diameter in the range of 0.5-3 nm.

In certain embodiments of the ligand-bound gold cluster (AuC) for use of manufacture of a medicament, the gold core has a diameter in the range of 0.5-2.6 nm.

In certain embodiments of the ligand-bound gold cluster (AuC) for use of manufacture of a medicament, the ligand is one selected from the group consisting of L-cysteine and its derivatives, D-cysteine and its derivatives, cysteine-containing oligopeptides and their derivatives, and other thiol-containing compounds.

In certain embodiments of the ligand-bound gold cluster (AuC) for use of manufacture of a medicament, the L-cysteine and its derivatives are selected from the group consisting of L-cysteine, N-isobutyryl-L-cysteine (L-NIBC), and N-acetyl-L-cysteine (L-NAC), and wherein the D-cysteine and its derivatives are selected from the group consisting of D-cysteine, N-isobutyryl-D-cysteine (D-NIBC), and N-acetyl-D-cysteine (D-NAC).

In certain embodiments of the ligand-bound gold cluster (AuC) for use of manufacture of a medicament, the cysteine-containing oligopeptides and their derivatives are cysteine-containing dipeptides, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

In certain embodiments of the ligand-bound gold cluster (AuC) for use of manufacture of a medicament, the cysteine-containing oligopeptides and their derivatives are cysteine-containing tripeptides, wherein the cysteine-containing tripeptides are selected from the group consisting of glycine-(D)L-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

In certain embodiments of the ligand-bound gold cluster (AuC) for use of manufacture of a medicament, the cysteine-containing oligopeptides and their derivatives are cysteine-containing tetrapeptides, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

In certain embodiments of the ligand-bound gold cluster (AuC) for use of manufacture of a medicament, the cysteine-containing oligopeptides and their derivatives are cysteine-containing pentapeptide, wherein the cysteine-containing pentapeptides are selected from the group consisting of Cysteine-Aspartic acid-Glutamic acid-Valine-Aspartic acid (CDEVD) and Aspartic acid-Glutamic acid-Valine-Aspartic acid-Cysteine (DEVDC).

In certain embodiments of the ligand-bound gold cluster (AuC) for use of manufacture of a medicament, the other thiol-containing compounds are selected from the group consisting of 1-[(2S)-2-methyl-3-thiol-1-oxopropyl]-L(D)-proline, thioglycolic acid, mercaptoethanol, thiophenol, D-3-trolovol, N-(2-mercaptopropionyl)-glycine, dodecyl mercaptan, 2-aminoethanethiol (CSH), 3-mercaptopropionic acid (MPA), and 4-mercaptobenoic acid (p-MBA).

The objectives and advantages of the invention will become apparent from the following detailed description of preferred embodiments thereof in connection with the accompanying drawings.

### DESCRIPTION OF THE DRAWINGS

Preferred embodiments according to the present invention will now be described with reference to the Figures, in which like reference numerals denote like elements.

FIG. 1 shows ultraviolet-visible (UV) spectrums, transmission electron microscope (TEM) images and particle size distribution diagrams of ligand L-NIBC-modified gold nanoparticles (L-NIBC-AuNPs) with different particle sizes; FIG. 1A shows the UV spectrum of 3.6 nm L-NIBC-AuNPs; FIG. 1B shows the TEM image of 3.6 nm L-NIBC-AuNPs; FIG. 1C shows the particle size distribution diagram of 3.6 nm L-NIBC-AuNPs; FIG. 1D shows the UV spectrum of 6.0 nm L-NIBC-AuNPs; FIG. 1E shows the TEM image of 6.0 nm L-NIBC-AuNPs; FIG. 1F shows the particle size distribution diagram of 6.0 nm L-NIBC-AuNPs; FIG. 1G shows the UV spectrum of 10.1 nm L-NIBC-AuNPs; FIG. 1H shows the TEM image of 10.1 nm L-NIBC-AuNPs; FIG. 1I shows the particle size distribution diagram of 10.1 nm L-NIBC-AuNPs; FIG. 1J shows the UV spectrum of 18.2 nm L-NIBC-AuNPs; FIG. 1K shows the TEM image of 18.2 nm L-NIBC-AuNPs; FIG. 1L shows the particle size distribution diagram of 18.2 nm L-NIBC-AuNPs.

FIG. 2 shows ultraviolet-visible (UV) spectrums, TEM images and particle size distribution diagrams of ligand L-NIBC-bound gold clusters (L-NIBC-AuCs) with different particle sizes; FIG. 2A shows the UV spectrum of 1.1 nm L-NIBC-AuCs; FIG. 2B shows the TEM image of 1.1 nm L-NIBC-AuCs; FIG. 2C shows the particle size distribution diagram of 1.1 nm L-NIBC-AuCs; FIG. 2D shows the UV spectrum of 1.8 nm L-NIBC-AuCs; FIG. 2E shows the TEM image of 1.8 nm L-NIBC-AuCs; FIG. 2F shows the particle size distribution diagram of 1.8 nm L-NIBC-AuCs; FIG. 2G shows the UV spectrum of 2.6 nm L-NIBC-AuCs; FIG. 2H shows the TEM image of 2.6 nm L-NIBC-AuCs; FIG. 2I shows the particle size distribution diagram of 2.6 nm L-NIBC-AuCs.

FIG. 3 shows infrared spectra of 1.1 nm, 1.8 nm and 2.6 L-NIBC-AuCs.

FIG. 4 shows UV, infrared, TEM, and particle size distribution diagrams of ligand CR-bound gold clusters (CR-AuCs); FIG. 4A shows UV spectrum of CR-AuCs; FIG. 4B shows infrared spectrum of CR-AuCs; FIG. 4C shows TEM image of CR-AuCs; FIG. 4D shows particle size distribution diagram of CR-AuCs.

FIG. 5 shows UV, infrared, TEM, and particle size distribution diagrams of ligand RC-bound gold clusters (RC-AuCs); FIG. 5A shows UV spectrum of RC-AuCs; FIG. 5B shows infrared spectrum of RC-AuCs; FIG. 5C shows TEM image of RC-AuCs; FIG. 5D shows particle size distribution diagram of RC-AuCs.

FIG. 6 shows UV, infrared, TEM, and particle size distribution diagrams of ligand 1-[(2S)-2-methyl-3-thiol-1-oxopropyl]-L-proline (i.e., Cap)-bound gold clusters (Cap-AuCs); FIG. 6A shows UV spectrum of Cap-AuCs; FIG. 6B shows infrared spectrum of Cap-AuCs; FIG. 6C shows TEM image of Cap-AuCs; FIG. 6D shows particle size distribution diagram of Cap-AuCs.

FIG. 7 shows UV, infrared, TEM, and particle size distribution diagrams of ligand GSH-bound gold clusters (GSH-AuCs); FIG. 7A shows UV spectrum of GSH-AuCs; FIG. 7B shows infrared spectrum of GSH-AuCs; FIG. 7C shows TEM image of GSH-AuCs; FIG. 7D shows particle size distribution diagram of GSH-AuCs.

FIG. 8 shows UV, infrared, TEM, and particle size distribution diagrams of ligand D-NIBC-bound gold clusters (D-NIBC-AuCs); FIG. 8A shows UV spectrum of D-NIBC-AuCs; FIG. 8B shows infrared spectrum of D-NIBC-AuCs; FIG. 8C shows TEM image of D-NIBC-AuCs; FIG. 8D shows particle size distribution diagram of D-NIBC-AuCs.

FIG. 9 shows UV, infrared, TEM, and particle size distribution diagrams of ligand L-cysteine-bound gold clusters (L-Cys-AuCs); FIG. 9A shows UV spectrum of L-Cys-AuCs; FIG. 9B shows infrared spectrum of L-Cys-AuCs; FIG. 9C shows TEM image of L-Cys-AuCs; FIG. 9D shows particle size distribution diagram of L-Cys-AuCs.

FIG. 10 shows UV, infrared, TEM, and particle size distribution diagrams of ligand 2-aminoethanethiol-bound gold clusters (CSH-AuCs); FIG. 10A shows UV spectrum of CSH-AuCs; FIG. 10B shows infrared spectrum of CSH-AuCs; FIG. 10C shows TEM image of CSH-AuCs; FIG. 10D shows particle size distribution diagram of CSH-AuCs.

FIG. 11 shows UV, infrared, TEM, and particle size distribution diagrams of ligand 3-mercaptopropionic acid-bound gold clusters (MPA-AuCs); FIG. 11A shows UV spectrum of MPA-AuCs; FIG. 11B shows infrared spectrum of MPA-AuCs; FIG. 11C shows TEM image of MPA-AuCs; FIG. 11D shows particle size distribution diagram of MPA-AuCs.

FIG. 12 shows UV, infrared, TEM, and particle size distribution diagrams of ligand 4-mercaptobenoic acid-bound gold clusters (p-MBA-AuCs); FIG. 12A shows UV spectrum of p-MBA-AuCs; FIG. 12B shows infrared spectrum of p-MBA-AuCs; FIG. 12C shows TEM image of p-MBA-AuCs; FIG. 12D shows particle size distribution diagram of p-MBA-AuCs.

FIG. 13 shows UV, TEM, and particle size distribution diagrams of ligand 4-Cysteine-Aspartic acid-Glutamic acid-Valine-Aspartic acid (CDEVD)-bound gold clusters (CDEVD-AuCs); FIG. 13A shows UV spectrum of CDEVD-AuCs; FIG. 13B shows TEM image of CDEVD-AuCs; FIG. 13C shows particle size distribution diagram of CDEVD-AuCs.

FIG. 14 shows UV, TEM, and particle size distribution diagrams of ligand 4-Aspartic acid-Glutamic acid-Valine-Aspartic acid-Cysteine (DEVDC)-bound gold clusters (DEVDC-AuCs); FIG. 14A shows UV spectrum of DEVDC-AuCs; FIG. 14B shows TEM image of DEVDC-AuCs; FIG. 14C shows particle size distribution diagram of DEVDC-AuCs.

FIG. 15 shows the neurological behavior scores of rats in each group (in the histogram of each time point, from left to right are sham operation group (blank), model control group, A1 low-dose group, A1 high-dose group, A2 low-dose group, A2 high-dose group, A3 low-dose group, A3 high-dose group, A4 low-dose group, A4 high-dose group, B1 low-dose group, B1 high-dose group, B2 low-dose group and B2 high-dose group).

FIG. 16 shows the percentage of cerebral infarction area of rats in each group (in the histogram, from left to right are sham operation group (blank), model control group, A1 low-dose group, A1 high-dose group, A2 low-dose group, A2 high-dose group, A3 low-dose group, A3 high-dose group, A4 low-dose group, A4 high-dose group, B1 low-dose group, B1 high-dose group, B2 low-dose group and B2 high-dose group).

FIG. 17 shows the exemplary TTC staining images of brain tissues in MCAO rats after administration of gold cluster drugs and gold nanoparticles; FIG. 17A shows the exemplary TTC staining image of sham operation group; FIG. 17B shows the exemplary TTC staining image of model control group; FIG. 17C shows the exemplary TTC staining image of A1 low-dose group; FIG. 17D shows the exemplary TTC staining image of A1 high-dose group; FIG. 17E shows the exemplary TTC staining image of B1 low-dose group; FIG. 17F shows the exemplary TTC staining image of B1 high-dose group.

FIG. 18 shows the results of mNSS neurological function scores at different time after completion of modeling, where NC: normal control group; SHAM: sham operation group; IVH: model control group; AL: Drug A low dose administration group; AH: Drug A high dose administration group; BL: Drug B low dose administration group; BH: Drug B high dose administration group. FIG. 18A shows the mNSS neurological function scores 12 hours after modeling; FIG. 18B shows the mNSS neurological function scores 1st Day after modeling; FIG. 18C shows the mNSS neurological function scores 2nd Day after modeling; FIG. 18D shows the mNSS neurological function scores 4th Day after modeling; FIG. 18E shows the mNSS neurological function scores 7th Day after modeling. ***: Compared with IVH group, P<0.001.

FIG. 19 shows content of Evans Blue in the supernatants of rat brain tissue homogenates of all groups in the measurement of permeability of blood-brain barrier (BBB) by Evans Blue Staining, where NC: normal control group; SHAM: sham operation group; IVH: model control group; AL: Drug A low dose administration group; AH: Drug A high dose administration group. *: Compared with IVH group rats, P<0.05; **: Compared with IVH group rats, P<0.01; ***: Compared with IVH group rats, P<0.001.

FIG. 20 shows water content of rat brain tissues measured by dry/wet weight method, where NC: normal control group; SHAM: sham operation group; IVH: model control group; AL: Drug A low dose administration group; AH: Drug A high dose administration group. *: Compared with IVH group rats, P<0.05; **: Compared with IVH group rats, P<0.01; ***: Compared with IVH group rats, P<0.001.

FIG. 21 shows exemplary H&E staining images of brain tissue in different groups; FIG. 21A: NC group; FIG. 21B: SHAM group; FIG. 21C: IVH group; FIG. 21D: AL group; FIG. 21E: AH group.

FIG. 22 shows exemplary images of iNOS immunofluorescence staining of rat brain tissues; FIG. 22A: NC group; FIG. 22B: SHAM group; FIG. 22C: IVH group; FIG. 22D: AL group; FIG. 22E: AH group.

FIG. 23 shows expression of MMP9 protein in brain tissue of rats in each group in WB experiment, where NC: normal control group; SHAM: sham operation group; IVH: model control group; AL: Drug A low dose administration group; AH: Drug A high dose administration group. FIG. 23A shows that MMP9 protein bands were detected in 6 parallel samples; FIG. 23B shows relative protein expression of MMP9 protein (GAPDH as reference). Compared with IVH group, *, P<0.05.

FIG. 24 shows MDA level in brain tissue of rats in each group, where NC: normal control group; SHAM: sham operation group; IVH: model control group; AL: Drug A low dose administration group; AH: Drug A high dose administration group. ***: Compared with IVH group, P<0.001; **: Compared with IVH group, P<0.01.

FIG. 25 shows SOD level in brain tissue of rats in each group, where NC: normal control group; SHAM: sham operation group; IVH: model control group; AL: Drug A low dose administration group; AH: Drug A high dose administration group. ***: Compared with IVH group, P<0.001.

FIG. 26 shows the results of transmission electron microscope examination of ultra-thin sections of brain tissue; FIG. 26A: NC group; FIG. 26B: SHAM group; FIG. 26C: IVH group; FIG. 26D: AL group; FIG. 26E: AH group.

### DETAILED DESCRIPTION OF THE EMBODIMENTS

The present invention may be understood more readily by reference to the following detailed description of certain embodiments of the invention.

Throughout this application, where publications are referenced, the disclosures of these publications are hereby incorporated by reference, in their entireties, into this application in order to more fully describe the state of art to which this invention pertains.

As used herein, “administering” means oral (“po”) administration, administration as a suppository, topical contact, intravenous (“iv”), intraperitoneal (“ip”), intramuscular (“im”), intralesional, intrahippocampal, intracerebroventricular, intranasal or subcutaneous (“sc”) administration, or the implantation of a slow-release device e.g., a mini-osmotic pump or erodible implant, to a subject. Administration is by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (i.e. other than through the alimentary tract, such as intramuscular, intravenous, intra-arterial, transdermal and subcutaneous) administration, with the proviso that, as used herein, systemic administration does not include direct administration to the brain region by means other than via the circulatory system, such as intrathecal injection and intracranial administration.

As used herein, the terms “treating” and “treatment” refer to delaying the onset of, retarding or reversing the progress of, or alleviating or preventing either the disease or condition to which the term applies, or one or more symptoms of such disease or condition.

The terms “patient,” “subject” or “individual” interchangeably refers to a mammal, for example, a human or a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster, guinea pig).

Gold clusters (AuCs) are a special form of gold existing between gold atoms and gold nanoparticles. AuCs have a size smaller than 3 nm, and are composed of only several to a few hundreds of gold atoms, leading to the collapse of face-centered cubic stacking structure of gold nanoparticles. As a result, AuCs exhibit molecule-like discrete electronic structures with distinct HOMO-LUMO gap unlike the continuous or quasi-continuous energy levels of gold nanoparticles. This leads to the disappearance of surface plasmon resonance effect and the corresponding plasmon resonance absorption band (520±20 nm) at UV-Vis spectrum that possessed by conventional gold nanoparticles.

The present invention provides a ligand-bound AuC.

In certain embodiments, the ligand-bound AuC comprises a ligand and a gold core, wherein the ligand is bound to the gold core. The binding of ligands with gold cores means that ligands form stable-in-solution complexes with gold cores through covalent bond, hydrogen bond, electrostatic force, hydrophobic force, van der Waals force, etc. In certain embodiments, the diameter of the gold core is in the range of 0.5-3 nm. In certain embodiments, the diameter of the gold core is in the range of 0.5-2.6 nm.

In certain embodiments, the ligand of the ligand-bound AuC is a thiol-containing compound or oligopeptide. In certain embodiments, the ligand bonds to the gold core to form a ligand-bonded AuC via Au-S bond.

In certain embodiments, the ligand is, but not limited to, L-cysteine, D-cysteine, or a cysteine derivative. In certain embodiments, the cysteine derivative is N-isobutyryl-L-cysteine (L-NIBC), N-isobutyryl-D-cysteine (D-NIBC), N-acetyl-L-cysteine (L-NAC), or N-acetyl-D-cysteine (D-NAC).

In certain embodiments, the ligand is, but not limited to, a cysteine-containing oligopeptide and its derivatives. In certain embodiments, the cysteine-containing oligopeptide is a cysteine-containing dipeptide. In certain embodiments, the cysteine-containing dipeptide is L(D)-cysteine-L(D)-arginine dipeptide (CR), L(D)-arginine-L(D)-cysteine dipeptide (RC), or L(D)-cysteine-L-histidine dipeptide (CH). In certain embodiments, the cysteine-containing oligopeptide is a cysteine-containing tripeptide. In certain embodiments, the cysteine-containing tripeptide is glycine-L(D)-cysteine-L(D)-arginine tripeptide (GCR), L(D)-proline-L(D)-cysteine-L(D)-arginine tripeptide (PCR), or L(D)-glutathione (GSH). In certain embodiments, the cysteine-containing oligopeptide is a cysteine-containing tetrapeptide. In certain embodiments, the cysteine-containing tetrapeptide is glycine-L(D)-serine-L(D)-cysteine-L(D)-arginine tetrapeptide (GSCR) or glycine-L(D)-cysteine-L(D)-serine-L(D)-arginine tetrapeptide (GCSR). In certain embodiments, the cysteine-containing oligopeptide is a cysteine-containing pentapeptide. In certain embodiments, the cysteine-containing pentapeptide is Cysteine-Aspartic acid-Glutamic acid-Valine-Aspartic acid (CDEVD), or Aspartic acid-Glutamic acid-Valine-Aspartic acid-Cysteine (DEVDC).

In certain embodiments, the ligand is a thiol-containing compound. In certain embodiments, thiol-containing compound is 1-[(2S)-2-methyl-3-thiol-1-oxopropyl]-L(D)-proline, thioglycollic acid, mercaptoethanol, thiophenol, D-3-trolovol, dodecyl mercaptan, 2-aminoethanethiol (CSH), 3-mercaptopropionic acid (MPA), or 4-mercaptobenoic acid (p-MBA).

The present invention provides a pharmaceutical composition for the treatment of cerebral stroke, where the cerebral stroke is selected from the group consisting of cerebral hemorrhagic stroke, cerebral ischemic stroke, and transient ischemic attack (TIA). In certain embodiments, the subject is human. In certain embodiments, the subject is a pet animal such as a dog.

In certain embodiments, the pharmaceutical composition comprises a ligand-bound AuC as disclosed above and a pharmaceutically acceptable excipient. In certain embodiments, the excipient is phosphate-buffered solution, or physiological saline.

The present invention provides a use of the above disclosed ligand-bound AuCs for manufacturing a medication for the treatment of cerebral stroke, where the cerebral stroke is selected from the group consisting of cerebral hemorrhagic stroke, cerebral ischemic stroke, and transient ischemic attack (TIA).

The present invention provides a use of the above disclosed ligand-bound AuCs for treating a subject with cerebral stroke, or a method for treating a subject with cerebral stroke using the above disclosed ligand-bound AuCs, where the cerebral stroke is selected from the group consisting of cerebral hemorrhagic stroke, cerebral ischemic stroke, and transient ischemic attack (TIA). In certain embodiments, the method for treatment comprises administering a pharmaceutically effective amount of ligand-bound AuCs to the subject. The pharmaceutically effective amount can be ascertained by routine in vivo studies. In certain embodiments, the pharmaceutically effective amount of ligand-bound AuCs is a dosage of at least 0.001 mg/kg/day, 0.005 mg/kg/day, 0.01 mg/kg/day, 0.05 mg/kg/day, 0.1 mg/kg/day, 0.5 mg/kg/day, 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 15 mg/kg/day, 20 mg/kg/day, 30 mg/kg/day, 40 mg/kg/day, 50 mg/kg/day, 60 mg/kg/day, 70 mg/kg/day, 80 mg/kg/day, or 100 mg/kg/day.

The following examples are provided for the sole purpose of illustrating the principles of the present invention; they are by no means intended to limit the scope of the present invention.

### EMBODIMENTS

### Embodiment 1. Preparation of Ligand-Bound AuCs

1.1 Dissolving HAuCl₄ in methanol, water, ethanol, n-propanol, or ethyl acetate to get a solution A in which the concentration of HAuCl₄ is 0.01˜0.03M;

1.2 Dissolving a ligand in a solvent to get a solution B in which the concentration of the ligand is 0.01˜0.18M; the ligand includes, but not limited to, L-cysteine, D-cysteine and other cysteine derivatives such as N-isobutyryl-L-cysteine (L-NIBC), N-isobutyryl-D-cysteine (D-NIBC), N-acetyl-L-cysteine (L-NAC), and N-acetyl-D-cysteine (D-NAC), cysteine-containing oligopeptides and their derivatives including, but not limited to, dipeptides, tripeptide, tetrapeptide, pentapeptide, and other peptides containing cysteine, such as L(D)-cysteine-L(D)-arginine dipeptide (CR), L(D)-arginine-L(D)-cysteine dipeptide (RC), L(D)-cysteine L(D)-histidine (CH), glycine-L(D)-cysteine-L(D)-arginine tripeptide (GCR), L(D)-proline-L(D)-cysteine-L(D)-arginine tripeptide (PCR), L(D)-glutathione (GSH), glycine-L(D)-serine-L(D)-cysteine-L(D)-arginine tetrapeptide (GSCR), glycine-L(D)-cysteine-L(D)-serine-L(D)-arginine tetrapeptide (GCSR), Cysteine-Aspartic acid-Glutamic acid-Valine-Aspartic acid pentapeptide (CDEVD) and Aspartic acid-Glutamic acid-Valine-Aspartic acid-Cysteine pentapeptide (DEVDC), and other thiol-containing compounds, such as one or more of 1-[(2S)-2-methyl-3-thiol-1-oxopropyl]-L(D)-proline, thioglycollic acid, mercaptoethanol, thiophenol, D-3-trolovol, dodecyl mercaptan, 2-aminoethanethiol (CSH), 3-mercaptopropionic acid (MPA), and 4-mercaptobenoic acid (p-MBA); the solvent is one or more of methanol, ethyl acetate, water, ethanol, n-propanol, pentane, formic acid, acetic acid, diethyl ether, acetone, anisole, 1-propanol, 2-propanol, 1-butanol, 2-butanol, pentanol, butyl acetate, tributyl methyl ether, isopropyl acetate, dimethyl sulfoxide, ethyl formate, isobutyl acetate, methyl acetate, 2-methyl-1-propanol and propyl acetate;

1.3 Mixing solution A and solution B so that the mole ratio between HAuCl₄ and ligand is 1:(0.01˜100), stirring them in an ice bath for 0.1-48 h, adding 0.025-0.8M NaBH₄ water, ethanol or methanol solution, continuing to stir in an ice water bath and react for 0.1-12 h. The mole ratio between NaBH₄ and ligand is 1:0.01˜100);

1.4 Using MWCO 3K˜30K ultrafiltration tubes to centrifuge the reaction solution at 8000˜17500 r/min by gradient for 10˜100 min after the reaction ends to obtain ligand-bound AuCs precipitate in different average particle sizes. The aperture of the filtration membranes for ultrafiltration tubes of different MWCOs directly decides the size of ligand-bound AuCs that can pass the membranes. This step may be optionally omitted;

1.5 Dissolving the ligand-bound AuCs precipitate in different average particle sizes obtained in step (1.4) in water, putting it in a dialysis bag and dialyzing it in water at room temperature for 1-7 days;

1.6 Freeze-drying ligand-bound AuCs for 12-24 h after dialysis to obtain a powdery or flocculant substance, i.e., ligand-bound AuCs.

As detected, the particle size of the powdery or flocculant substance obtained by the foregoing method is smaller than 3 nm (distributed in 0.5-2.6 nm in general). No obvious absorption peak at 520 nm. It is determined that the obtained powder or floc is ligand-bound AuCs.

### Embodiment 2. Preparation and Characterization of AuCs Bound with Different Ligands

2.1 Preparation of L-NIBC-bound AuCs, i.e. L-NIBC-AuCs

Taking ligand L-NIBC for example, the preparation and confirmation of AuCs bound with ligand L-NIBC are detailed.

2.1.1 Weigh 1.00 g of HAuCl₄ and dissolve it in 100 mL of methanol to obtain a 0.03M solution A;

2.1.2 Weigh 0.57 g of L-NIBC and dissolve it in 100 mL of glacial acetic acid (acetic acid) to obtain a 0.03M solution B;

2.1.3 Measure 1 mL of solution A, mix it with 0.5 mL, 1 mL, 2 mL, 3 mL, 4 mL, or 5 mL of solution B respectively (i.e. the mole ratio between HAuCl₄ and L-NIBC is 1:0.5, 1:1, 1:2, 1:3, 1:4, 1:5 respectively), react in an ice bath under stirring for 2 h, quickly add 1 mL of freshly prepared 0.03M (prepared by weighing 11.3 mg of NaBH₄ and dissolving it in 10 mL of ethanol) NaBH₄ ethanol solution when the solution turns colorless from bright yellow, continue the reaction for 30 min after the solution turns dark brown, and add 10 mL of acetone to terminate the reaction.

2.1.4 After the reaction, the reaction solution is subjected to gradient centrifugation to obtain L-NIBC-AuCs powder with different particle sizes. Specific method: After the reaction is completed, the reaction solution is transferred to an ultrafiltration tube with MWCO of 30K and a volume of 50 mL, and centrifuged at 10000 r/min for 20 min, and the retentate in the inner tube is dissolved in ultrapure water to obtain powder with a particle size of about 2.6 nm. Then, the mixed solution in the outer tube is transferred to an ultrafiltration tube with a volume of 50 mL and MWCO of 10K, and centrifuged at 13,000 r/min for 30 min. The retentate in the inner tube is dissolved in ultrapure water to obtain powder with a particle size of about 1.8 nm. Then the mixed solution in the outer tube is transferred to an ultrafiltration tube with a volume of 50 mL and MWCO of 3K, and centrifuged at 17,500 r/min for 40 min. The retentate in the inner tube is dissolved in ultrapure water to obtain powder with a particle size of about 1.1 nm.

2.1.5 Precipitate the powder in three different particle sizes obtained by gradient centrifugation, remove the solvent respectively, blow the crude product dry with N2, dissolve it in 5 mL of ultrapure water, put it in a dialysis bag (MWCO is 3 KDa), put the dialysis bag in 2 L of ultrapure water, change water every other day, dialyze it for 7 days, freeze-dry it and keep it for future use.

2.2 Characterization of L-NIBC-AuCs

Characterization experiment was conducted for the powder obtained above (L-NIBC-AuCs). Meanwhile, ligand L-NIBC-modified gold nanoparticles (L-NIBC-AuNPs) are used as control. The method for preparing gold nanoparticles with ligand being L-NIBC refers to the reference (W. Yan, L. Xu, C. Xu, W. Ma, H. Kuang, L. Wang and N. A. Kotov, Journal of the American Chemical Society 2012, 134, 15114; X. Yuan, B. Zhang, Z. Luo, Q. Yao, D. T. Leong, N. Yan and J. Xie, Angewandte Chemie International Edition 2014, 53, 4623).

2.2.1 Observation of the morphology by transmission electron microscope (TEM)

The test powders (L-NIBC-AuCs sample and L-NIBC-AuNPs sample) were dissolved in ultrapure water to 2 mg/L as samples, and then test samples were prepared by hanging drop method. More specifically, 5 μL of the samples were dripped on an ultrathin carbon film, volatized naturally till the water drop disappeared, and then observe the morphology of the samples by JEM-2100F STEM/EDS field emission high-resolution TEM.

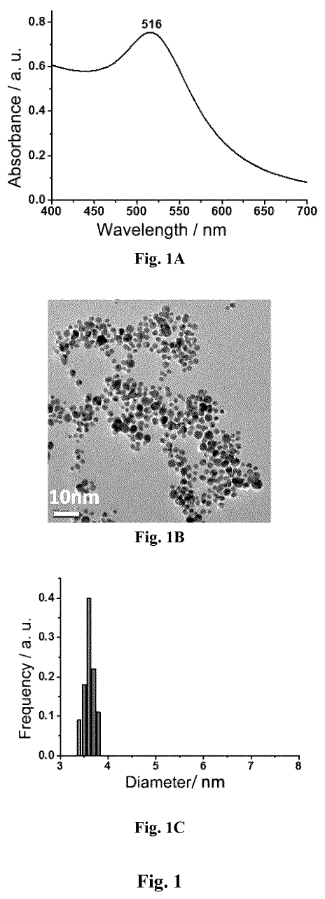

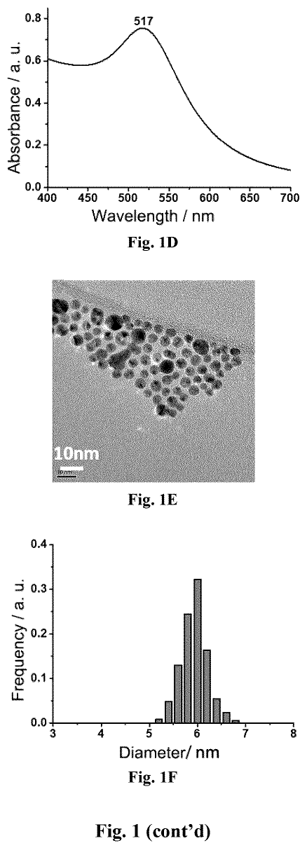

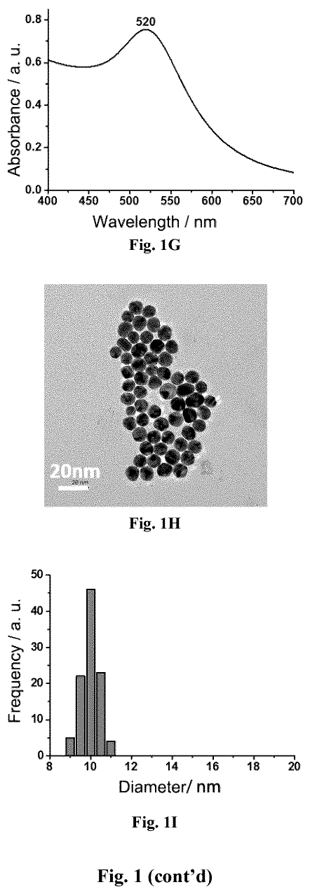

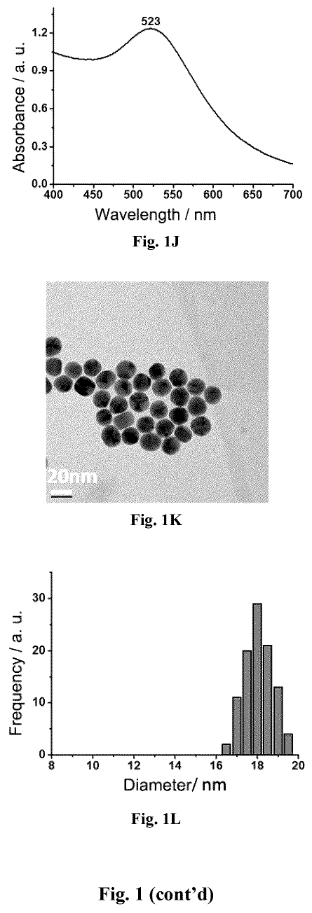

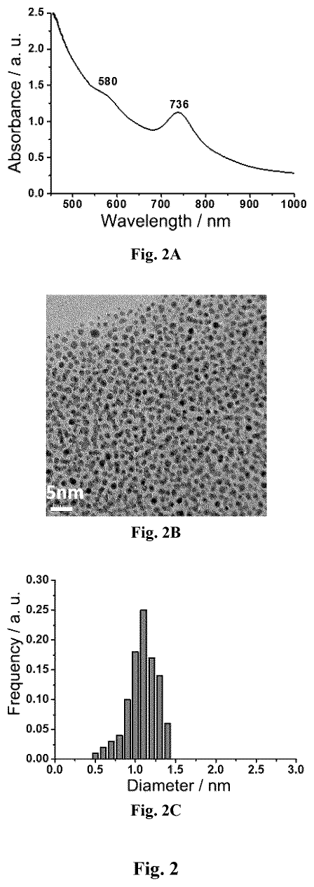

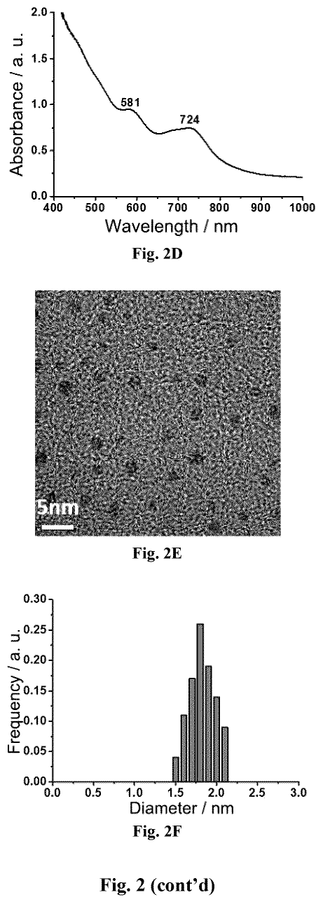

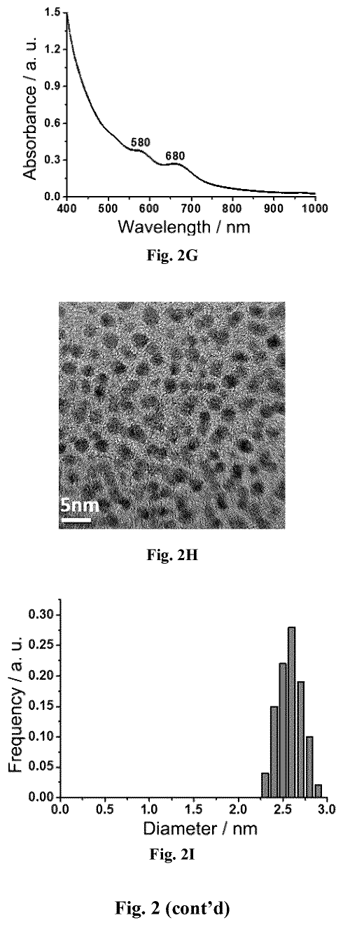

The four TEM images of L-NIBC-AuNPs are shown in FIG. 1B, FIG. 1E, FIG. 1H, and FIG. 1K of FIG. 1; the three TEM images of L-NIBC-AuCs are shown in FIG. 2B, FIG. 2E, and FIG. 2H of FIG. 2.

The images in FIG. 2 indicate that each of L-NIBC-AuCs samples has a uniform particle size and good dispersibility, and the average diameter of L-NIBC-AuCs (refer to the diameter of gold core) is 1.1 nm, 1.8 nm and 2.6 nm respectively, in good accordance with the results in FIG. 2C, FIG. 2F, and FIG. 2I of FIG. 2. In comparison, L-NIBC-AuNPs samples have a larger particle size. Their average diameter (refer to the diameter of gold core) is 3.6 nm, 6.0 nm, 10.1 nm and 18.2 nm respectively, in good accordance with the results in FIG. 1C, FIG. 1F, FIG. 11, and FIG. 1L of FIG. 1.

2.2.2 Ultraviolet (UV)-visible (vis) absorption spectra

The test powders (L-NIBC-AuCs sample and L-NIBC-AuNPs sample) were dissolved in ultrapure water till the concentration was 10 mg·L⁻¹, and the UV-vis absorption spectra were measured at room temperature. The scanning range was 190-1100 nm, the sample cell was a standard quartz cuvette with an optical path of 1 cm, and the reference cell was filled with ultrapure water.

The UV-vis absorption spectra of the four L-NIBC-AuNPs samples with different sizes are shown in FIG. 1A, FIG. 1D, FIG. 1G, and FIG. 1J of FIG. 1, and the statistical distribution of particle size is shown in FIG. 1C, FIG. 1F, FIG. 1I, and FIG. 1L of FIG. 1; the UV-vis absorption spectra of three L-NIBC-AuCs samples with different sizes are shown in FIG. 2A, FIG. 2D, and FIG. 2G of FIG. 2, and the statistical distribution of particle size is shown in FIG. 2C, FIG. 2F, and FIG. 2I of FIG. 2.

FIG. 1 indicates that due to the surface plasmon effect, L-NIBC-AuNPs had an absorption peak at about 520 nm. The position of the absorption peak is relevant with particle size. When the particle size is 3.6 nm, the UV absorption peak appears at 516 nm; when the particle size is 6.0 nm, the UV absorption peak appears at 517 nm; when the particle size is 10.1 nm, the UV absorption peak appears at 520 nm, and when the particle size is 18.2 nm, the absorption peak appears at 523 nm. None of the four samples has any absorption peak above 560 nm.

FIG. 2 indicates that in the UV absorption spectra of three L-NIBC-AuCs samples with different particle sizes, the surface plasmon effect absorption peak at 520 nm disappeared, and two obvious absorption peaks appeared above 560 nm and the positions of the absorption peaks varied slightly with the particle sizes of AuCs. This is because AuCs exhibit molecule-like properties due to the collapse of the face-centered cubic structure, which leads to the discontinuity of the density of states of AuCs, the energy level splitting, the disappearance of plasmon resonance effect and the appearance of a new absorption peak in the long-wave direction. It could be concluded that the three powder samples in different particle sizes obtained above are all ligand-bound AuCs.

2.2.3 Fourier transform infrared spectroscopy

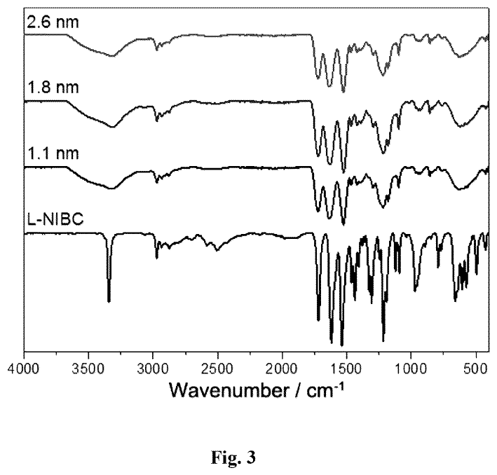

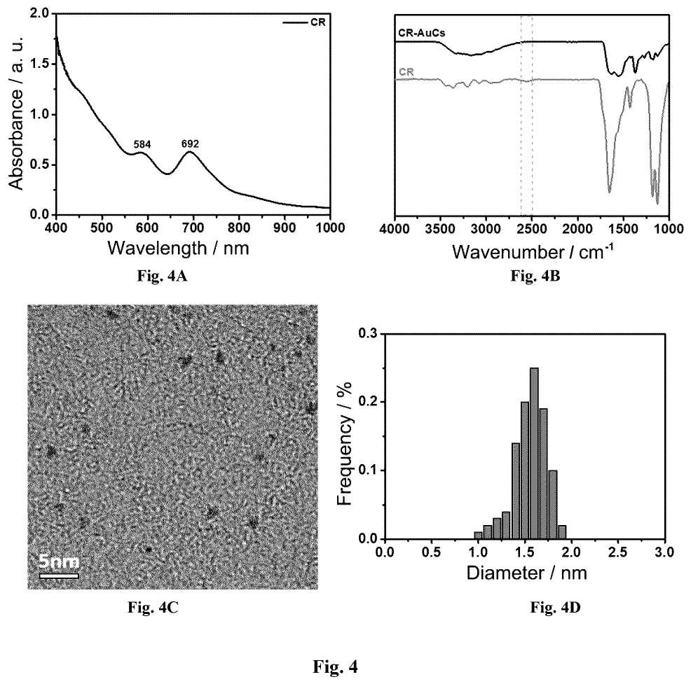

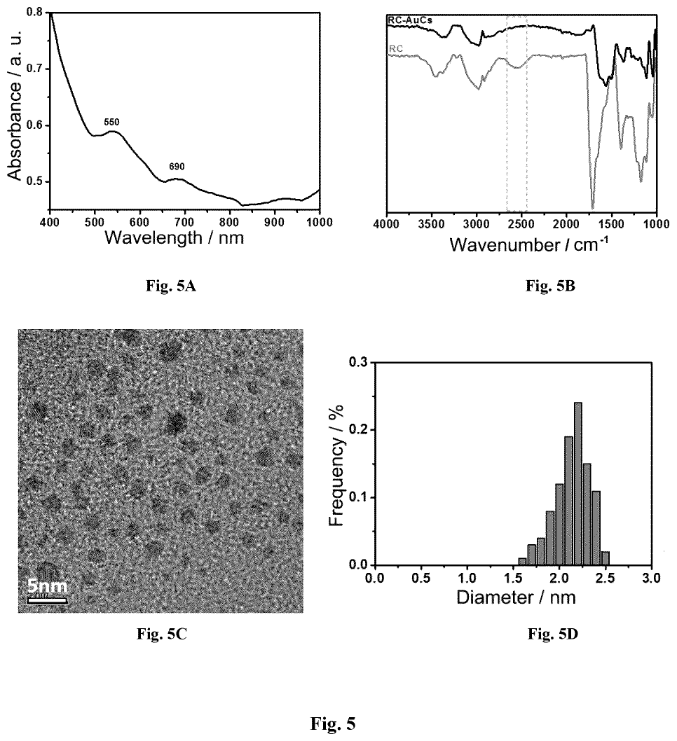

Infrared spectra were measured on a VERTEX80V Fourier transform infrared spectrometer manufactured by Bruker in a solid powder high vacuum total reflection mode. The scanning range is 4000-400 cm⁻¹ and the number of scans is 64. Taking L-NIBC-AuCs samples for example, the test samples were L-NIBC-AuCs dry powder with three different particle sizes and the control sample was pure L-NIBC powder. The results are shown in FIG. 3.

FIG. 3 shows the infrared spectrum of L-NIBC-AuCs with different particle sizes. Compared with pure L-NIBC (the curve at the bottom), the S-H stretching vibrations of L-NIBC-AuCs with different particle sizes all disappeared completely at 2500-2600 cm⁻¹, while other characteristic peaks of L-NIBC were still observed, proving that L-NIBC molecules were successfully bound to the surface of AuCs via Au-S bond. The figure also shows that the infrared spectrum of the ligand-bound AuCs is irrelevant with its size.

AuCs bound with other ligands were prepared by a method similar to the above method, except that the solvent of solution B, the feed ratio between HAuCl₄ and ligand, the reaction time and the amount of NaBH₄ added were slightly adjusted. For example: when L-cysteine, D-cysteine, N-isobutyryl-L-cysteine (L-NIBC) or N-isobutyryl-D-cysteine (D-NIBC) is used as the ligand, acetic acid is selected as the solvent; when dipeptide CR, dipeptide RC or 1-[(2S)-2-methyl-3-thiol-1-oxopropyl]-L-proline is used as the ligand, water is selected as the solvent, and so on and so forth; other steps are similar, so no further details are provided herein.

The present invention prepared and obtained a series of ligand-bound AuCs by the foregoing method. The ligands and the parameters of the preparation process are shown in Table 1.

TABLE 1
Preparation parameters of AuCs bound with different ligands in the present invention

| | | Parameter | | | | | | |
|---|---|---|---|---|---|---|---|---|
| | | | | | | | Time of | |
| | | | | | | | reaction | |
| | | | | Time of | | | in an ice | |
| | | | | reaction in | | | bath | |
| | | | | an ice bath | | Mole | under | |
| | | | | under | | ratio | stirring | |
| | | | Feed ratio | stirring | | between | after | |
| | | | between | before | | HAuCl₄ | addition | |
| | | Solvent used | HAuCl₄ and | addition | | and | of | |
| | Ligand | for solution B | ligand | of NaBH₄ | | NaBH₄ | NaBH₄ | |
| 1 | L-cysteine | Acetic acid | 1:3 | 2 | h | 1:2 | 0.5 | h |
| 2 | D-cysteine | Acetic acid | 1:3 | 2 | h | 1:2 | 0.5 | h |
| 3 | N-acetyl-L-cysteine | Ethanol | 1:4 | 1 | h | 1:1 | 0.5 | h |
| 4 | N-acetyl-D-cysteine | Ethanol | 1:4 | 1 | h | 1:1 | 0.5 | h |
| 5 | L-NIBC | Water | 1:4 | 0.5 | h | 1:2 | 0.5 | h |
| 6 | D-NIBC | Water | 1:4 | 0.5 | h | 1:2 | 0.5 | h |
| 7 | Other cysteine | Soluble | 1:(0.1~100) | 0.5 h~24 h | | 1:(0.1~100) | 0.1~24 | h |
| | derivatives | solvent | | | | | | |
| 8 | CR | Water | 1:4 | 22 | h | 2:1 | 0.5 | h |
| 9 | RC | Water | 1:4 | 20 | h | 2:1 | 0.5 | h |
| 10 | HC | Water | 1:3 | 12 | h | 1:2 | 2 | h |
| 11 | CH | Ethanol | 1:4 | 16 | h | 1:3 | 3 | h |
| 12 | GSH | Water | 1:2 | 12 | h | 1:1 | 3 | h |
| 13 | KCP | Water | 1:3 | 15 | h | 1:2 | 1 | h |
| 14 | PCR | Water | 1:4 | 16 | h | 1:3 | 2 | h |
| 15 | GSCR | Water | 1:4 | 16 | h | 1:3 | 1.5 | h |
| 16 | GCSR | Water | 1:3 | 12 | h | 1:2 | 2 | h |
| 17 | CDEVD | Water | 1:7 | 1 | h | 1:0.1 | 0.5 | h |
| 18 | DEVDC | Water | 1:7 | 1 | h | 1:0.1 | 0.5 | h |
| 19 | Other oligopeptides | Soluble | 1:(0.1~100) | 0.5 h~24 h | | 1:(0.1~100) | 0.1~24 | h |
| | containing cysteine | solvent | | | | | | |
| 20 | 1-[(2S)-2-methyl-3- | Water | 1:8 | 2 | h | 1:7 | 1 | h |
| | thiol-1-oxopropyl]-L- | | | | | | | |
| | proline | | | | | | | |
| 21 | Mercaptoethanol | Ethanol | 1:2 | 2 | h | 1:1 | 1 | h |
| 22 | Thioglycollic acid | Acetic acid | 1:2 | 2 | h | 1:1 | 1 | h |
| 23 | Thiophenol | Ethanol | 1:5 | 5 | h | 1:1 | 1 | h |
| 24 | D-3-trolovol | Water | 1:2 | 2 | h | 1:1 | 1 | h |
| 25 | N-(2- | Water | 1:2 | 2 | h | 1:1 | 1 | h |
| | mercaptopropionyl)- | | | | | | | |
| | glycine | | | | | | | |
| 26 | Dodecyl mercaptan | Methanol | 1:5 | 5 | h | 1:1 | 1 | h |
| 27 | 2-aminoethanethiol | Water | 1:5 | 2 | h | 8:1 | 0.5 | h |
| | (CSH) | | | | | | | |
| 28 | 3-mercaptopropionic | Water | 1:2 | 1 | h | 5:1 | 0.5 | h |
| | acid (MPA) | | | | | | | |
| 29 | 4-mercaptobenoic | Water | 1:6 | 0.5 | h | 3:1 | 2 | h |

| | acid (p-MBA) | | | | | | |
|---|---|---|---|---|---|---|---|
| 30 | Other compounds | Soluble | 1:(0.01~100) | 0.5 h~24 h | 1:(0.1~100) | 0.1~24 | h |

| | |
|---|---|
| containing thiol | solvent |

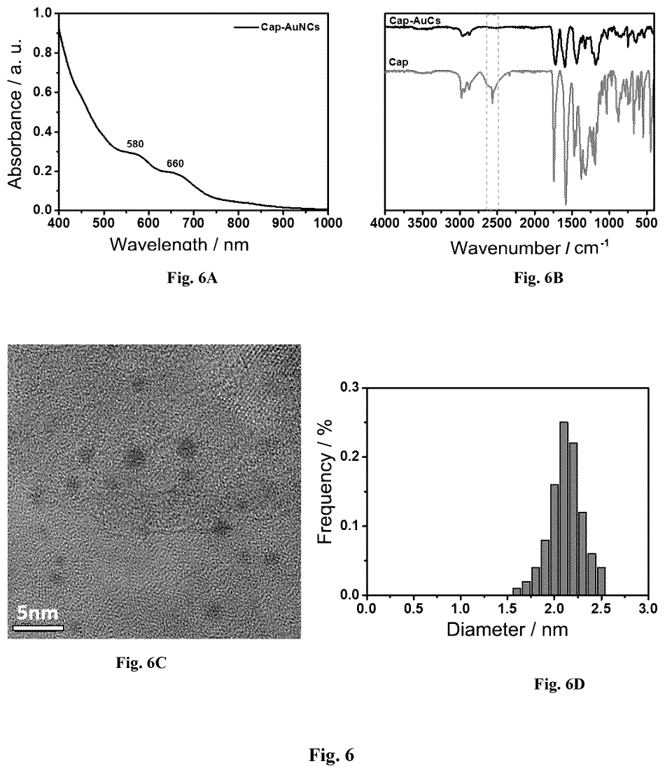

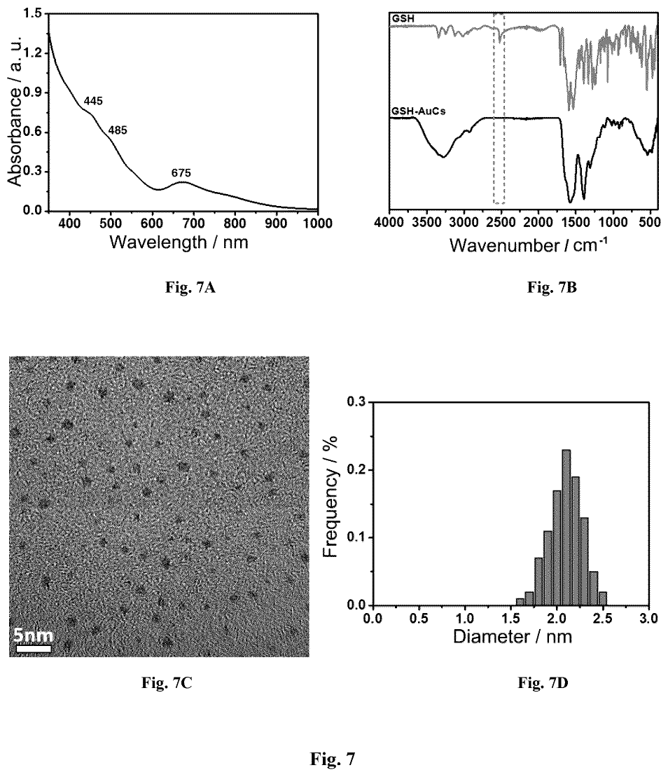

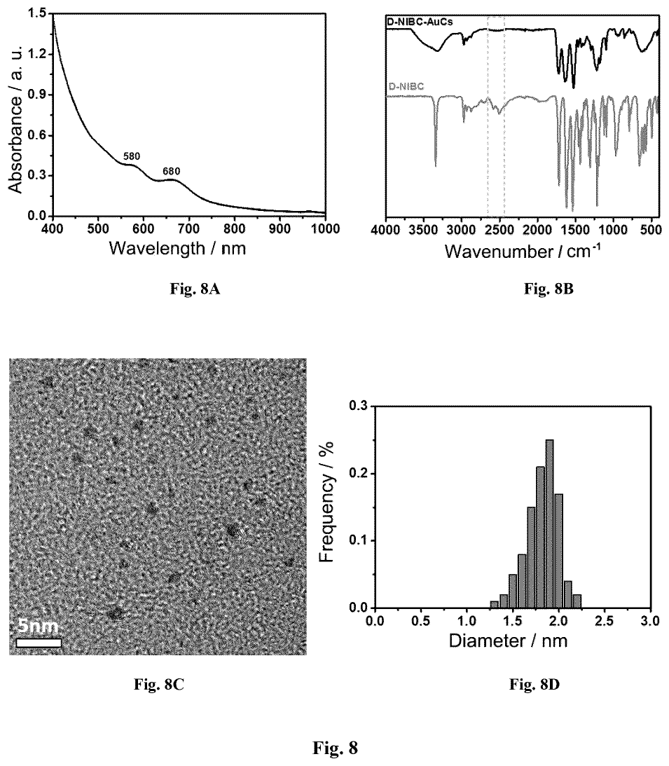

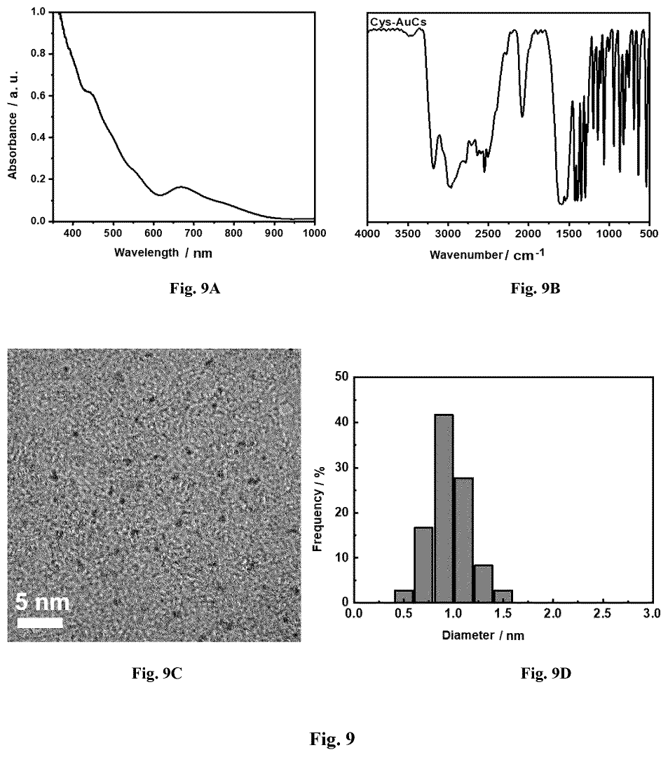

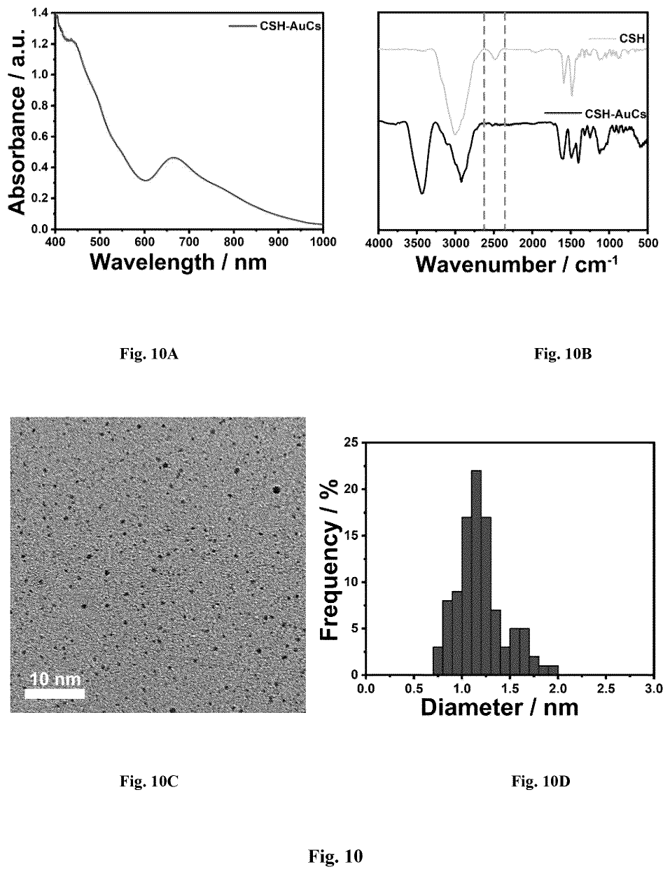

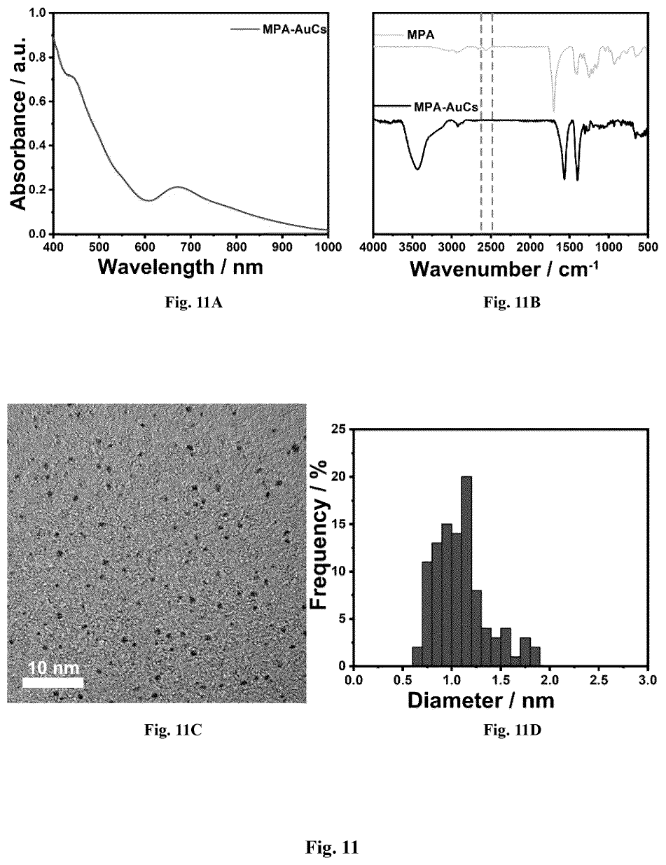

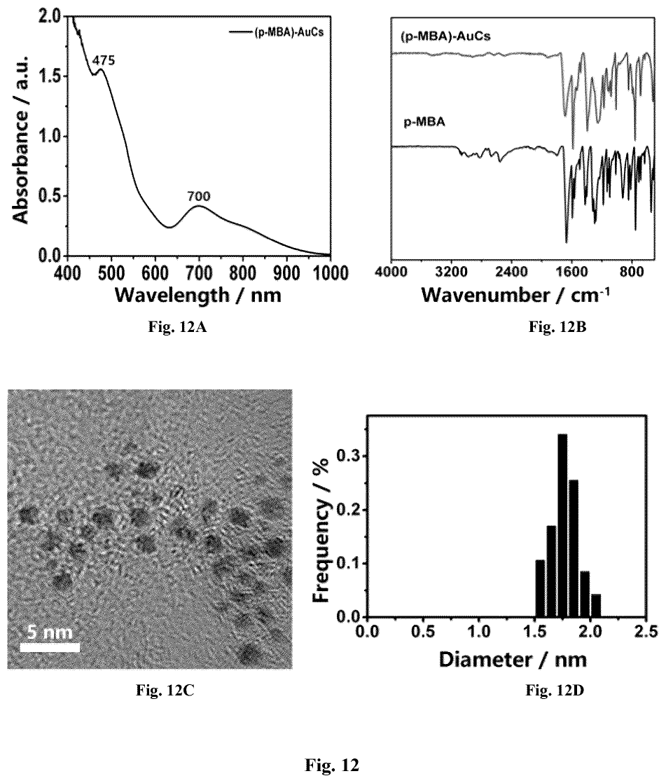

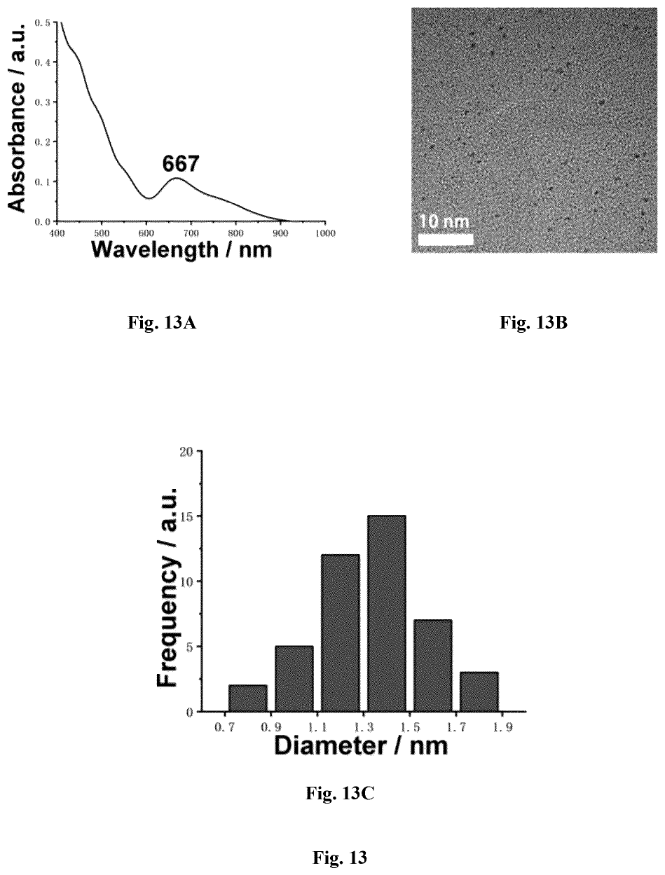

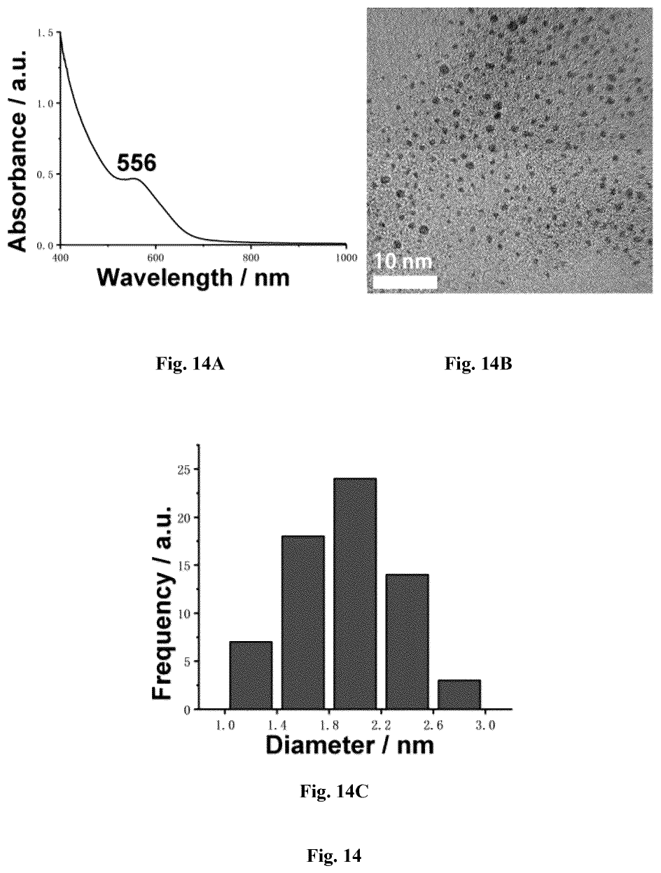

The samples listed in Table 1 are confirmed by the foregoing methods. The characteristics of eleven (11) different ligand-bound AuCs are shown in FIG. 4 (CR-AuCs), in FIG. 5 (RC-AuCs), in FIG. 6 (Cap-AuCs) (Cap denotes 1-[(2S)-2-methyl-3-thiol-1-oxopropyl]-L-proline), in FIG. 7 (GSH-AuCs), in FIG. 8 (D-NIBC-AuCs), in FIG. 9 (L-Cys-AuCs), in FIG. 10 (CSH-AuCs), in FIG. 11 (MPA-AuCs), in FIG. 12 (p-MBA-AuCs), in FIG. 13 (CDEVD-AuCs), and in FIG. 14 (DEVDC-AuCs). FIG. 4A-FIG. 12A show UV spectra; FIG. 4B-FIG. 12B show infrared spectra; FIG. 4C-FIG. 12C show TEM images; and FIG. 4D-FIG. 12D show particle size distribution. FIG. 13A and FIG. 14A show UV spectra; FIG. 13B and FIG. 14B show TEM images; FIG. 13C and FIG. 14C show particle size distribution.

The results indicate that the diameters of AuCs bound with different ligands obtained from Table 1 are all smaller than 3 nm. Ultraviolet spectra also show disappearance of peak at 520±20 nm, and appearance of absorption peak in other positions. The position of the absorption peak could vary with ligands and particle sizes as well as structures. In certain situations, there is no special absorption peak, mainly due to the formation of AuCs mixtures with different particles sizes and structures or certain special AuCs that moves the position of absorption peak beyond the range of UV-vis spectrum. Meanwhile, Fourier transform infrared spectra also show the disappearance of ligand thiol infrared absorption peak (between the dotted lines in FIG. 4B-FIG. 8B), while other infrared characteristic peaks are all retained, suggesting that all ligand molecules have been successfully bound to gold atoms to form ligand-bound AuCs, and the present invention has successfully obtained AuCs bound with the ligands listed in Table 1.

### Embodiment 3. Cerebral Ischemic Stroke Animal Model Experiments

3.1 Testing Samples

Gold clusters:

A1: ligand L-NIBC-bound gold clusters (L-NIBC-AuCs), size distribution in the range of 0.5-3.0 nm;

A2: ligand L-cysteine-bound gold clusters (L-Cys-AuCs), size distribution in the range of 0.5-3.0 nm;

A3: ligand N-acetyl-L-cysteine-bound gold clusters (L-NAC-AuCs), size distribution in the range of 0.5-3.0 nm; and

A4: ligand DEVDC-bound gold clusters (DEVDC-AuCs), size distribution in the range of 0.5-3.0 nm.

Gold nanoparticles:

B1: L-NIBC-bound gold nanoparticles (L-NIBC-AuNPs), size distribution range of 6.1±1.5 nm; and

B2: L-NAC-bound gold nanoparticles (L-NAC-AuNPs), size distribution range of 9.0±2.4 nm.

All testing samples were prepared following the above-described method with slight modification, and their quality was characterized using the above described methods.

3.2 Experimental protocols

3.2.1 Establishment of rat middle cerebral artery occlusion (MCAO) model and administration of test substances

Male SPF grade Sprague Dawley (SD) rats (220-260 g) were purchased from Shanghai Shrek Experimental Animal Co., Ltd. All rats were acclimatized to the environment for 7 days prior to the experiments. Rats were randomly divided into 14 groups (n=10), including sham operation group, model control group, low (2 mg/kg rat body weight) and high-dose group (10 mg/kg rat body weight) of gold cluster drugs A1, A2, A3 and A4, and low (2 mg/kg rat body weight) and high-dose group (10 mg/kg rat body weight) of gold nanoparticle B1 and B2. On the day of the experiments, the rats were anesthetized with 10% chloral hydrate (350 mg/kg body weight). The right common carotid artery, internal carotid artery and external carotid artery were exposed through the midline incision. The suture was inserted into the internal carotid artery (ICA) 18 mm±0.5 mm through the external carotid artery (ECA), until the MCA regional blood supply was blocked, resulting in cerebral infarction. After 1.5 h, the suture was withdrawn to the entrance of ECA for reperfusion. The basic cerebral blood flow (CBF) before operation and after embolization were measured by flow meter. The animals whose CBF decreased continuously (rCBF≥70%) were considered to be successful models of middle cerebral artery occlusion (MCAO). After reperfusion, the rats were injected intraperitoneally with drugs or solvents (normal saline) at 0 h, 24 h, 48 h and 72 h respectively. The neurological behavior scores were evaluated at 0 h, 24 h, 48 h, 72 h and 96 h. The experiment was terminated at 96 h after operation. Brain collection and TTC staining were performed after euthanasia. Images of brain slices were taken and the percentage of cerebral infarction area was calculated.

3.2.2 Neurological behavior score

0 point: no difference from normal rats; 1 point: right front paw extension is not straight, head to the opposite side; 2 points: walking discontinuous circles in the open space; 3 points: walking continuous circles in the open space; 4 points, unconscious walking, collapse to one side; 5 points: death.

3.2.3 Infarct area (TTC staining)

The rats were euthanized by carbon dioxide inhalation. The brains were taken and put into the brain trough for coronal section (2 mm). Staining was with 2% TTC in dark at room temperature. After taking photos, the infarct area was analyzed by ImageJ. The percentage of infarct area (%)=(contralateral hemisphere area−(ipsilateral hemisphere area−infarction area))/contralateral hemisphere area×100%.

3.2.4 Statistical analysis

Statistical analysis was performed by Graph Pad Prism Software 7.0 (CA, US). The data were expressed as mean±standard error, and the statistical analysis was performed by Dunnett test. P<0.05 denotes statistically significant.

3.3 Results

3.3.1 cerebral blood flow in the cerebral ischemic region

More than 70% decrease of rat cerebral blood flows (reduction cerebral blood flow, rCBF≥70%) indicates successful establishment of MACO model. Except for the sham operation group, all remaining groups had rCBF more than 70%, with an average of about 80%, demonstrating successful establishment of MCAO model.

3.3.2 Effects of each drug on rat neurological behavior

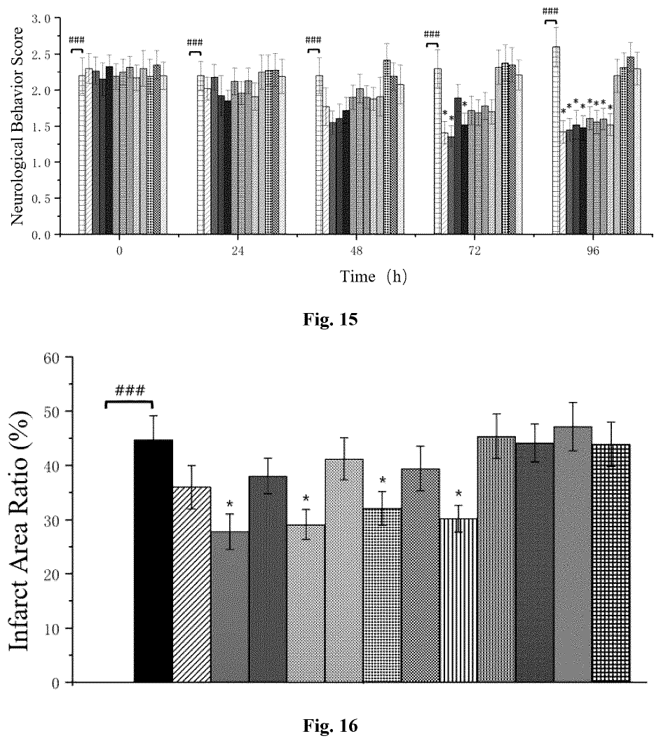

FIG. 15 shows the neurological behavior scores of rats in each group (in the histogram of each time point, from left to right are sham operation group (blank), model control group, A1 low-dose group, A1 high-dose group, A2 low-dose group, A2 high-dose group, A3 low-dose group, A3 high-dose group, A4 low-dose group, A4 high-dose group, B1 low-dose group, B1 high-dose group, B2 low-dose group and B2 high-dose group). The rats in the sham operation group had normal neurological behavior, and the behavior score was 0; the rats in the model control group showed severe behavioral functional defects at 0 h, 24 h, 48 h, 72 h and 96 h after operation (compared with the sham operation group, P<0.001, ###). Compared with the model control group, the neurological behavior scores of A1, A2, A3, A4 low-dose groups and high-dose groups had no significant improvement at 24 hours after operation. At 48 h post operation, the neurological behavior scores of A1, A2, A3 and A4 low-dose groups and high-dose groups began to decline, but there was no statistical difference (compared with the model control group, P>0.05). At 72 h post operation, the neurological behavior scores of the four drugs were further decreased, among which A1 low-dose group, A1 high-dose group and A2 high-dose group showed significant differences (compared with model control group, P<0.05, *). At 96 h post operation, there were significant differences for all the low-dose groups and the high-dose groups of the four drugs (compared with the model control group, P<0.05, *). These results suggest that all four gold cluster drugs can significantly improve the neurological behavior deficits induced by ischemic stroke, and the effect is dose-dependent to a certain extent.

Compared with the model control group, the low and high dose groups of gold nanoparticles B1 and B2 did not significantly improve the neurological behavior scores of MACO model rats at 24 h, 48 h, 72 h and 96 h after operation, indicating that gold nanoparticles could not significantly improve the behavioral disorders caused by cerebral ischemic stroke.

3.3.3.3 Effect of each drug on cerebral infarction areas of MACO model rats

FIG. 16 shows the percentage of cerebral infarction area of rats in each group (in the histogram, from left to right are sham operation group (blank), model control group, A1 low-dose group, A1 high-dose group, A2 low-dose group, A2 high-dose group, A3 low-dose group, A3 high-dose group, A4 low-dose group, A4 high-dose group, B1 low-dose group, B1 high-dose group, B2 low-dose group and B2 high-dose group). In the sham operation group, the brain tissue was normal and no infarction occurred; the infarct area was 0%. The infarct area of the model control group was 44.7%±4.5% (P<0.001, ###). Compared with the model control group, the percentages of cerebral infarction areas in A1, A2, A3, A4 low and high-dose groups were evidently decreased, but there was no significant difference in the low-dose groups, while significant difference was found in the high-dose groups (compared with model control group, P<0.05, *). Taking A1 as an example, the infarct area of the low-dose group decreased from 44.7±4.5% to 36.0±4.0% (compared with model control group, P>0.05), while that of high-dose group decreased to 27.8±3.4% (compared with model control group, P<0.05, *).

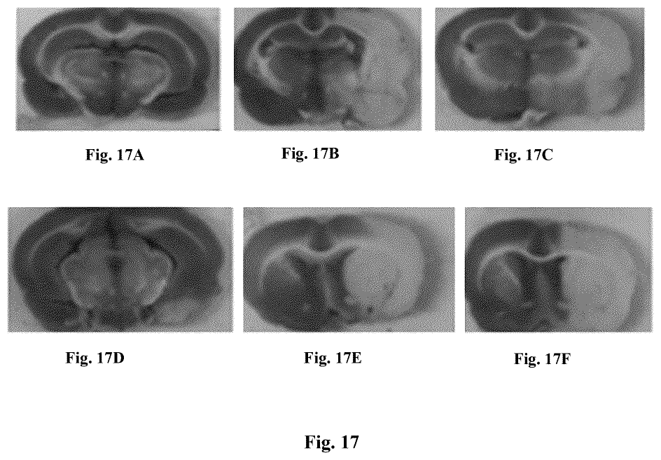

FIG. 17 presents the exemplary images of TTC staining brain tissues of MCAO rats after administration of the gold clusters drugs represented by A1 and gold nanoparticles represented by B1. In FIG. 17, FIG. 17A: sham operation group; FIG. 17B: model control group; FIG. 17C: A1 low-dose group; FIG. 17D: A1 high-dose group; FIG. 17E: B1 low-dose group; FIG. 17F: B1 high-dose group. As can be seen from FIG. 17, the rats in the sham operation group did not have cerebral infarction, while the model control group had a large cerebral infarction (white part on the right). The area of cerebral infarction after low-dose administration of A1 drug was reduced (the white part on the right side was reduced), while the area of cerebral infarction was significantly reduced by high-dose administration of A1 drug (the white part on the right side was greatly reduced), while the low-dose and high-dose administration of B1 had no effect on the area of cerebral infarction (the white part on the right side had no reduction). A2, A3 and A4 showed similar effect to A1 in reducing infarct area, while B2 was similar to B1 with no reduction of infarct area.

Other ligand-bound AuCs also have the similar effects on treating cerebral ischemic stroke, while their effects vary to certain extents. They would not be described in detail here.

### Embodiment 4. Cerebral Hemorrhagic Stroke Animal Model Experiments

4.1 Materials and methods

4.1.1 Test drugs

Drug A: L-NIBC-modified gold nanoclusters (L-NIBC-AuCs), the diameter of gold cores in the range of 0.5-3.0 nm;

Drug B: L-Cys-modified gold nanoclusters (L-Cys-AuCs), the diameter of gold cores in the range of 0.5-3.0 nm.

4.1.2 Animals and groupings

140 SD female rats (8-weeks old, 190-220 g) were adaptively reared in SPF for 7 days and then randomly divided into 7 groups:
(1) Normal control (NC) group, 20 rats, average body weight 214.0±5.1 g;
(2) Sham operation (SHAM) group, 20 rats, average body weight 213.5±6.5 g;
(3) Model control (IVH) group, 20 rats, average body weight 214.7±6.1 g;
(4) Drug A low dose (4 mg/kg body weight) administration (AL) group, 20 rats, average body weight 212.5±7.3 g;
(5) Drug A high dose (10 mg/kg body weight) administration (AH) group, 20 rats, average body weight 212.1±6.8 g;
(6) Drug B low dose (4 mg/kg body weight) administration (BL) group, 20 rats, average body weight 213.2±6.3 g; and
(7) Drug B high dose (10 mg/kg body weight) administration (BH) group, 20 rats, average body weight 211.2±7.1 g.

There was no significant difference in body weight among the groups.

4.1.3 Modeling, drug administration, and test protocol

Establishing intraventricular hemorrhage (IVH) model in rats:

Rats in IVH group and each drug administration group were anesthetized by an intraperitoneal injection of 2% barbiturate (50 mg/kg). After anesthesia, the head skin was disinfected; rats were fixed on a stereo locator in a prone position. By adjusting the stereo locator, rat incisors were fixed on the incisor hook, so that the anterior and posterior fontanels were at the same level. Then the ear rod was adjusted and fixed to make sure that the head of rats did not move. After disinfecting the head skin and furs of rats, the skin was incised in the middle of rat head, and the periosteum was denuded with 3% hydrogen peroxide, exposing the anterior and posterior fontanels and the coronal suture. A small hole about 1 mm in diameter was drilled with a dental drill 0.2 mm behind the anterior fontanel and 3 mm beside the midline without damaging the dura mater and brain tissue. Then, the rat tail was cleaned with 40° C. warm water. After hyperemia, the rat tail was disinfected with ethanol, and 50 μl of non-anticoagulant arterial blood was taken by using a 1 ml syringe. The syringe with blood was fixed onto the stereo locator. The micro syringe was inserted to a depth of 6 mm through the hole, and the autologous arterial blood was injected twice; injecting 10 μl arterial blood evenly; stopping injecting blood for 2 minutes; then injecting 40 μl arterial blood evenly and slowly again; letting the needle stay for 4 minutes; withdrawing the needle about 2.0 mm; stopping the needle again for 4 minutes; and then slowly withdrawing the syringe completely. After suturing the incision, sterile cotton ball was used to compress and stop bleeding. The rats were checked daily to observe wound healing and any possible infection.

The rat behavior was observed after rats woke up. The mNSS neurological function score was used to evaluate rat modeling. After operation, drug was daily administered by intraperitoneal injection at day 1, day 2, day 3, day 4, day 5, day 6 and day 7, where the dosage for AL group and AH group was 4 and 10 mg/kg body weight respectively. Rats in NC group and SHAM group were intraperitoneally injected with the same volume of normal saline at corresponding time points. All rats were evaluated with mNSS score at 12 h, 24 h, 3 d, 5 d and 7 d after intraperitoneal injections.

4.1.4. Measurement of the permeability of blood-brain barrier by Evans Blue staining

The treated rats were injected intravenously with Evans Blue Stain (0.5%), and the eyes and skin of the rats appeared blue. After 0.5-1 hour, sacrificed the rats and taken out their brain tissues. Put the brain tissue into a 1.5 ml centrifuge tube, added 1 ml PBS, quickly homogenized the brain tissue with a tissue homogenizer and centrifuged. Taken the supernatant, added the same amount of trichloroacetic acid and incubated at 4° C. Centrifuged for 15 min. Taken the supernatant and measured the absorbance value (OD value) at 620 nm with a spectrophotometer. At the same time, the OD values of standard Evans Blue with known different gradients were measured and the standard curve was drawn. Calculated the Evans Blue content of the samples to be tested according to the standard curve.

4.1.5. Measurement of brain water content

Dry/wet weight method is adopted. 24 hours after ischemia-reperfusion, the rats were subjected to excessive anesthesia. After decapitation, taken the brain, removed the olfactory bulb, cerebellum and low brain stem, separated the left and right cerebral hemispheres, weighed immediately to record their wet weight; then dried the brain tissues in an electric oven at 110° C. for 24 hours, and then weighed the dried brain tissues quickly to record their dry weight. Calculated the brain water content (%)=(wet weight-dry weight)/wet weight×100%.

4.1.6. H&E staining

Brain tissues were taken out and placed in embedding boxes. Steps of dehydration, transparency, wax immersion, embedding, slicing and baking were sequentially carried out, and then H & E staining was performed: taking out the slices from the oven, treating the slices with xylene twice for 15 minutes each time, then treating the slices with 100%, 95%, 80% and 70% ethanol respectively for 5 minutes, and then treating the slices with ultrapure water twice for 5 minutes each time to complete the dewaxing step; staining the slices with hematoxylin dye for 5 minutes and rinsing the slices with ultrapure water for 3 times; after cell nuclei turned into blue by washing with tap water for 10 minutes, staining the slices with 0.5% eosin dye for 5 minutes, washing the slices with tap water for 5 times, drying the slices in a 60° C. oven, and finally sealing the slices with neutral resin. Optical microscope was used to observe and collect images under 200 times field of vision.

4.1.7 Immunofluorescence (IF) staining

Rat brain tissues were taken out and placed in embedding boxes. The steps of dehydration, transparency, wax immersion, embedding, slicing and baking were sequentially performed. The slices were taken out from the oven and dewaxed. Then the steps of antigen repair, cleaning, permeability, iNOS first antibody treatment, goat anti-rabbit second antibody treatment, and DAPI staining were carried out. The final steps were drying and sealing. Fluorescence microscope was used to observe and collect images under 200 times field of vision.

4.1.8 Western-blot (WB)

First, protein samples were prepared. Taking out frozen tissues from refrigerator, weighing an appropriate amount of the tissue, adding the lysis buffer containing protease inhibitor according to the ratio of 1:9, shaking at 4° C. until all the tissue pieces were broken. Placing the samples on ice for 20 min, centrifuging at 12000 rpm at 4° C. for 20 min, and taking the supernatant. BCA protein concentration assay kit was used to determine the protein concentration of each sample. Adjusting the protein concentration according to the results of concentration determination to ensure the consistency of protein concentration among different groups. After boiling at 95° C. for 5 min, the sample was loaded, and the remaining samples were stored at −80° C.

Then performing WB according to the following steps:
Preparation of electrophoretic gel: after cleaning and drying glass plates, fixing them on a gel maker; then starting to prepare separation gel, pouring the separation gel into the gap of the glass plates to a proper height, and covering the separation gel with anhydrous ethanol until the gel was completely polymerized. Pouring out anhydrous ethanol, washing it gently with double distilled water, and then sucking up to dry with filter paper. Then adding concentration gel to an appropriate height and inserting the comb. After the concentration gel was completely polymerized, taking out the comb.
Electrophoresis: the processed samples were loaded in a total amount of 40 μg per well. The electrophoresis was carried out with 80V for concentration gel, 120V for separation gel and constant voltage power supply; the position of target proteins was determined according to the relative position of the molecular weight of pre-staining markers and the target proteins. When the target protein was in the best resolution position of the lower ⅓ of the separation gel, the electrophoresis separation was stopped.
Membrane transfer: soaking cut PVDF membranes in methanol for 10 s, and then putting them into a new membrane transfer solution for use. Taking out the gel, cutting the target strip according to Marker, rinsing with distilled water; cutting the same size PVDF film and filter paper with the PAGE gel, and soaking the PVDF film and filter paper in the electrotransfer buffer. According to the order of black plate-fiber mat-filter paper-gel-PVDF membrane-filter paper-fiber mat-white plate, placing them sequentially, clamping the plates, putting it into the membrane transfer apparatus, connecting the black plate side to black negative pole. Filling up the electrotransfer tank with membrane transfer buffer and starting the membrane transfer. The process was carried out at 4° C. (ice bath), and the conditions were 200 mA, 90 min (0.45 μm membrane) or 200 mA, 60 min (0.2 μm membrane).
Blocking: The membranes were washed with TBST for 3 times and then were blocked with TBST blocking solution containing 5% milk for 2 h on shaking table at room temperature.
Primary antibody: diluting the corresponding primary antibody with TBST containing 3% BSA, immersing the PVDF membrane in the primary antibody incubation solution, and incubating overnight at 4° C. MMP9 was diluted with 1:1000 and iNOS with 1:2000. After incubation, washing the PVDF membrane with TBST for 3 times, 10 min/time, to remove the excess primary antibody.
Secondary antibody: diluting HRP-labeled secondary antibody (1:5000) with blocking solution, immersing the PVDF membrane in the secondary antibody incubation solution, and incubating at room temperature for 1 h. After incubation, washing the PVDF membrane was washed with TBST for 3 times, 10 min/time, to remove the excess secondary antibody.
Exposure: mixing the enhancement solution with stable peroxidase solution in the ratio of 1:1 from the ECL kit to prepare working solution, dropping an appropriate amount of the working solution on the PVDF film, and exposing it with automatic chemiluminescence image analysis system.
Regeneration of membrane by elution: after exposure, washing the PVDF membrane with TBST for 3 times, 5 min/time, immersing the PVDF membrane in a membrane regeneration solution by adding an appropriate amount of the membrane regeneration solution, and eluting on a shaking table at room temperature for 20 minutes. After elution, fully washing the PVDF membrane with TBST for 3 times, 5 min/time, to remove the excess membrane regeneration solution.

The specific process for the second blocking, internal control incubation, secondary antibody binding and exposure was the same as the above experimental process, where both β-Tubulin and GAPDH were diluted at the ratio of 1:5000.

The results of the exposure were analyzed by Image J software.

4.1.9 Assay of lipid peroxidation

Preparation of samples: adding PBS into tissues (w/v: 1:9), homogenizing fully, incubating on ice for 10 min, centrifuging at 4000 rpm for 10 min, and taking the supernatant for assay.

Malondialdehyde (MDA) kit was used for assay. Blank tube, standard tube, measuring tube and control tube were prepared according to instructions. The absorbance (OD) value at 532 nm was detected. The content of MDA in the supernatant was calculated as follows: MDA content (nmol/ml)=(measured OD value−control OD value)/(standard OD value−Blank OD value)×Standard concentration (10 nmol/ml).

4.1.10 Assay of Superoxide dismutase (SOD) index

Preparation of samples: adding PBS into tissues (w/v: 1:9), homogenizing fully, incubating on ice for 10 min, centrifuging at 4000 rpm for 10 min, and taking the supernatant for assay.

SOD kit was used for assay according to the instructions. Calculating according to the following formula: SOD inhibition rate (%)=((OD value of control well−OD value of control blank well)−(OD value of measuring well−OD value of measuring blank well))/(OD value of control well−OD value of control blank well)×100%; SOD activity (U/mgprot)=SOD inhibition rate÷50%× Dilution ratio of reaction system÷protein concentration of sample (mgprot/ml).

4.1.11 Electron microscopic examination

Taking tissue blocks about 1 cubic millimeter. After fixation, dehydration, embedding and curing, the tissue blocks were sliced with ultra-thin slicer into slices with a thickness of 70 nm. Then, double-staining the slices with 2% uranyl acetate-lead citrate. Transmission electron microscope was used to observe and take photos.

4.2 Results

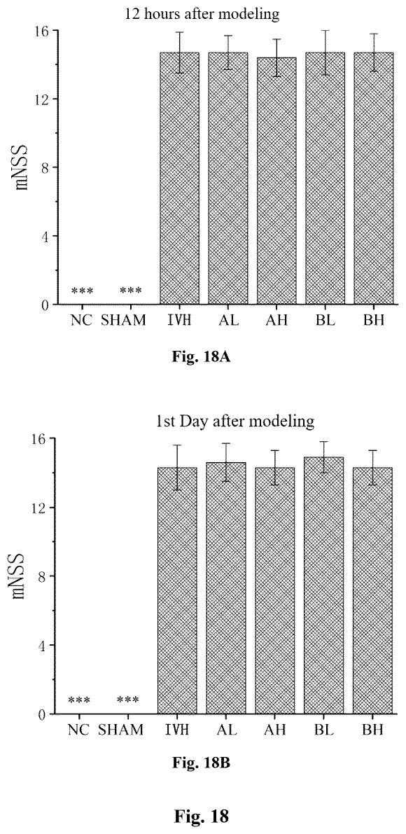

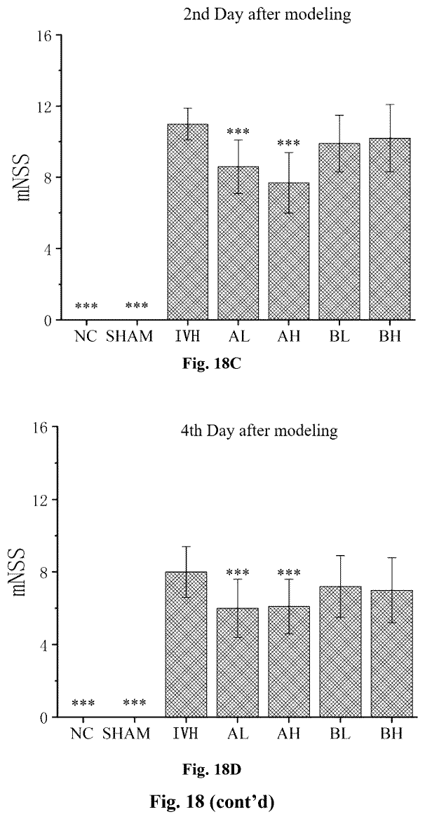

FIG. 18 shows the results of mNSS neurological function scores at different time after completion of modeling, where NC: normal control group; SHAM: sham operation group; IVH: model control group; AL: Drug A low dose administration group; AH: Drug A high dose administration group; BL: Drug B low dose administration group; BH: Drug B high dose administration group.

As shown in FIG. 18A, the results showed that 12 hours after modeling, the average mNSS scores of neurological functions are: normal control (NC) group (0.0±0.0), sham operation (SHAM) group (0.0±0.0), model control (IVH) group (14.7±1.2), drug A administration low dose (AL) group (14.7±1.0), drug A administration high dose (AH) group (14.4±1.1) and drug B administration low dose (BL) group (14.7±1.3), drug B administration high dose (BH) group (14.7±1.1). Compared with IVH group, there was significant difference for NC group and SHAM group (P<0.001, ***), while there was no significant difference for other groups compared with IVH group.

As shown in FIG. 18B, the results showed that the first day after modeling, the average mNSS scores of neurological functions are: NC group (0.0±0.0), SHAM group (0.0±0.0), IVH group (14.3±1.3), AL group (14.6±1.1), AH group (14.3±1.0), BL group (14.9±0.9), BH group (14.3±1.0). Compared with IVH group, there was significant difference for NC group and SHAM group and IVH group (P<0.001, ***), while there was no significant difference for other groups compared with IVH group.

As shown in FIG. 18C, the results showed that the second day after modeling, the average mNSS scores of neurological functions are: NC group (0.0±0.0), SHAM group (0.0±0.0), IVH group (11.0±0.9), AL group (8.6±1.5), AH group (7.7±1.7), BL group (9.9±1.8), BH group (10.2±1.9). Compared with IVH group, there was significant difference for NC group, SHAM group, AL group and AH group (P<0.001, **). However, compared with IVH group, BL and BH groups showed no significant difference even though their mNSS scores are lower than that of IVH group.

As shown in FIG. 18D, the results showed that the fourth day after modeling, the average mNSS scores of neurological functions are: NC group (0.0±0.0), SHAM group (0.0±0.0), IVH group (8.0±1.5), AL group (6.0±1.8), AH group (6.1±1.7), BL group (7.2±1.8), BH group (7.0±1.8). Compared with IVH group, BL and BH groups showed no significant difference, but all remaining groups showed significant difference (P<0.001, **).

As shown in FIG. 18E, the results showed that the seventh day after modeling, the average mNSS scores of neurological functions are: NC group (0.0±0.0), SHAM group (0.0±0.0), IVH group (5.4±1.7), AL group (2.9±0.9), AH group (2.8±1.0), BL group (4.5±1.8), BH group (4.1±1.6). Compared with IVH group, BL and BH groups showed no significant difference, but all remaining groups showed significant difference (P<0.001, **).

These results indicate that different doses of drug A can significantly improve the mNSS neurological function score of IVH rats from the second day after modeling, indicating that drug A has excellent effect in the treatment of IVH rats. However, from the second day, the mNSS score of drug B was lower than that of IVH group, but there was no significant difference (P>0.05), indicating that drug B was not effective in the treatment of IVH model rats.

In order to further study the therapeutic effect of drug A on IVH model rats, we carried out the research from multiple perspectives.

FIG. 19 shows the results of Evans Blue Staining for measuring the permeability of blood-brain barrier (BBB). It can be seen that after Evans Blue Staining, the content of Evans Blue in IVH group was significantly higher than that in NC and SHAM groups (P<0.001, ***). The contents of Evans Blue in brain tissues of rats in the low dose administration (AL) group and high dose administration (AH) group were significantly lower than that in IVH group (P<0.05, *; P<0.01, **). The results show that IVH modeling leads to a significant increase in the permeability of blood-brain barrier in rats, and both Drug A low-dose and high-dose administrations can significantly improve the increased permeability of blood-brain barrier caused by IVH modeling.

Brain edema caused by IVH modeling is an important cause of increased intracranial pressure, brain hernia and animal death. Using dry/wet weight method to detect water content of rat brain tissue can evaluate the situation of rat brain edema. FIG. 20 shows the results of water content in rat brain tissue measured by dry/wet weight method. Through analysis, it was found that the water contents of brain tissues in NC group and SHAM group were significantly lower than that in IVH model rats (P<0.001, ***), and the water contents of brain tissues in Drug A low-dose administration (AL) group and high-dose administration (AH) group were also significantly lower than that in IVH group (P<0.05, *; P<0.01, **). These results show that both Drug A low-dose and high-dose administrations can significantly improve the brain edema caused by IVH modeling, so as to improve the prognosis of animals.

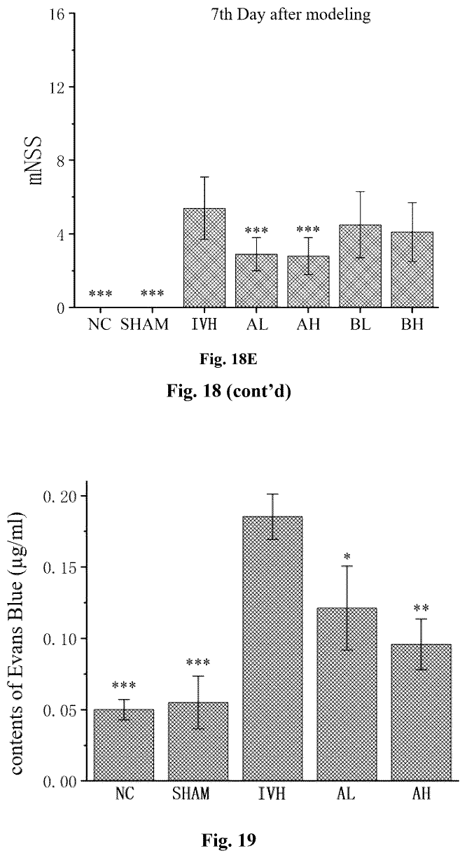

FIG. 21 shows exemplary images of H&E staining. The results showed that there were some gaps around the blood vessels and cells in NC group (FIG. 21A), slight injury in SHAM group (FIG. 21B), and obvious enlargement of the gaps around the blood vessels and cells in IVH group, with cellular degeneration and vacuoles, indicating severe brain injury (FIG. 21C). Drug A administration low dose (AL) (FIG. 21D) and high dose (AH) (FIG. 21E) can significantly reduce the brain injury of IVH model rats.

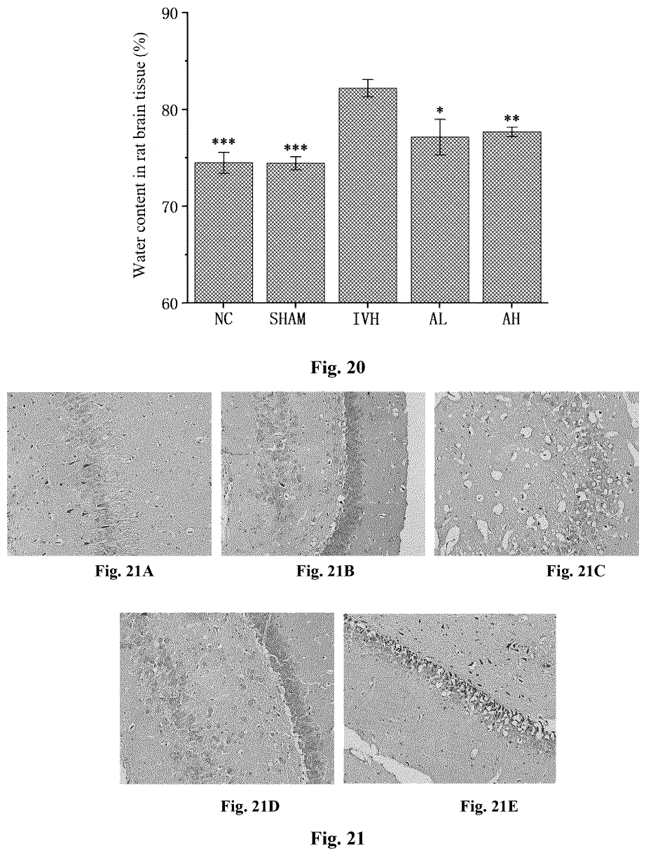

Injury-induced nitric oxide synthase (iNOS) is expressed after brain injury. FIG. 22 shows exemplary images of iNOS immunofluorescence staining in rat brain tissues. It can be seen that the positive expression of iNOS in IVH group (FIG. 22C) was relatively higher. The expression of iNOS in other groups, including NC group (FIG. 22A), SHAM group (FIG. 22B), AL group (FIG. 22D) and AH group (FIG. 22E), was decreased in varying degrees compared with that in IVH group. This indicates that the expression of iNOS in rat brain tissue is increased after IVH modeling, and the drug A administration low and high doses can reduce the expression of iNOS, exerting protective effect on brain injury.

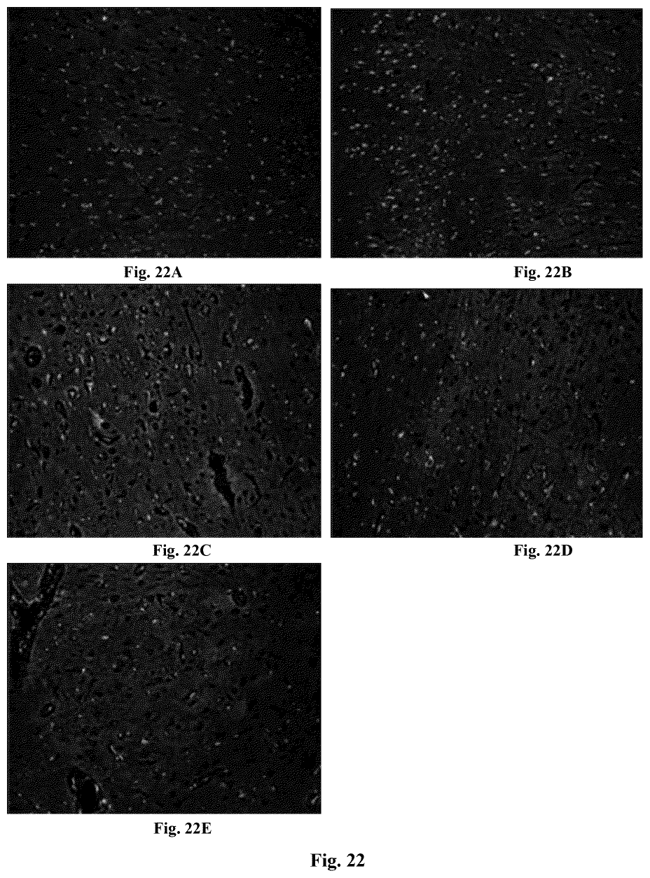

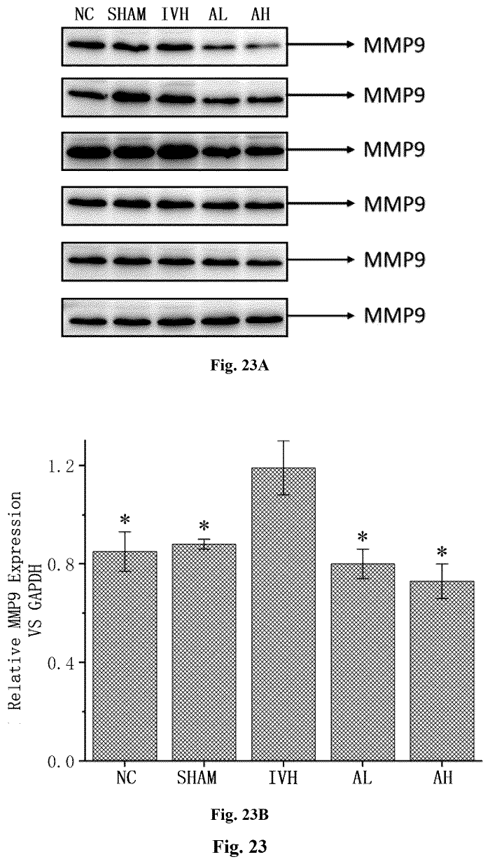

Gelatinase B (MMP9) is no or low expression in normal brain tissue. However, after brain injury caused by IVH modeling, MMP9 may play an important role in the process of angiogenic brain edema and other brain injury by destroying the blood-brain barrier. FIG. 23 shows the expression of MMP9 protein in the brain tissue of rats in each group. FIG. 23A shows the MMP9 protein bands of 6 parallel samples obtained from WB test, and FIG. 23B shows the relative protein expression of MMP9 (with GAPDH as the reference). It can be seen that the relative expression of MMP9 in the brain tissue of NC group, SHAM group, AL group and AH group was significantly lower than that of IVH group (P<0.05, *). These results indicate that both low and high doses of drug A can significantly reduce the increased expression of MMP9 protein induced by brain injury in model rats, indicating that drug A has a protective effect on brain injury.

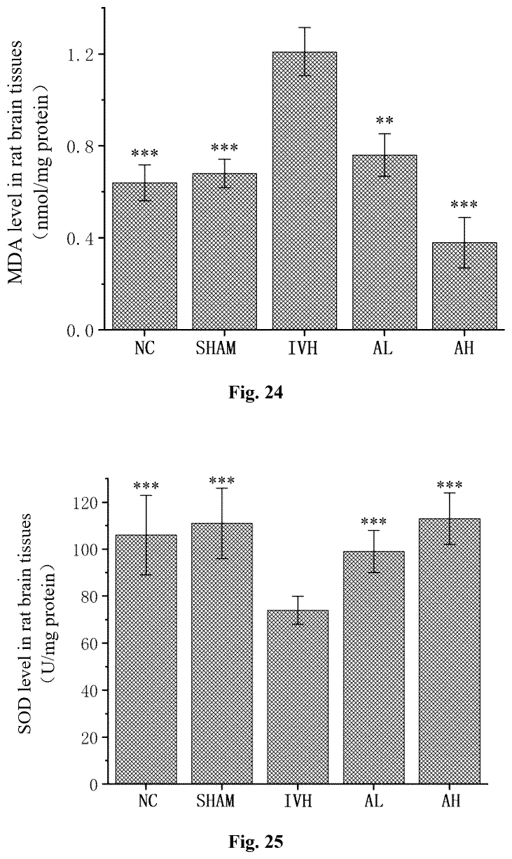

Malondialdehyde (MDA) is the end product of peroxidation reaction between free radicals and unsaturated fatty acids of cell membrane. It is an important biomarker of lipid oxidative damage. It can indirectly reflect the degree of tissue peroxidation damage. Its level is closely related to the severity of clinical symptoms in the pathogenesis of stroke, and has important clinical significance in the diagnosis, treatment and prognosis of stroke. FIG. 24 shows the expression of MDA in brain tissue of rats in each group. It can be seen that the MDA expression in NC, SHAM, AL and AH groups is significantly lower than that of IVH group, indicating that the brain injury resulted from IVH modeling causes a significant increase of MDA expression in the brain (P<0.001, *** in NC group and SHAM group compared with IVH group), while low and high doses of drug A administration can significantly reduce the MDA expression pertinent to brain injury (compared with IVH group, P<0.01, ** and 0.001, ***, respectively).

Superoxide dismutase (SOD) is the most effective free radical scavenging enzyme in the body. The SOD produced by the body can reflect the situation of free radicals in the body. Some studies have shown that the expression and activity of SOD in hemorrhagic stroke brain tissue are significantly lower than those in normal brain tissue. FIG. 25 shows the expression level of SOD in brain tissue of rats in each group. The results showed that the expression level of SOD of NC, SHAM, AL and AH groups were significantly higher than that of IVH group, indicating that the brain injury resulted from IVH modeling caused significant decrease of SOD expression in the brain (compared with IVH group, both NC group and SHAM group: P<0.001, ***), while low and high doses of drug A administration can significantly increase SOD expression in the brain (compared with IVH group, P<0.001, ***).

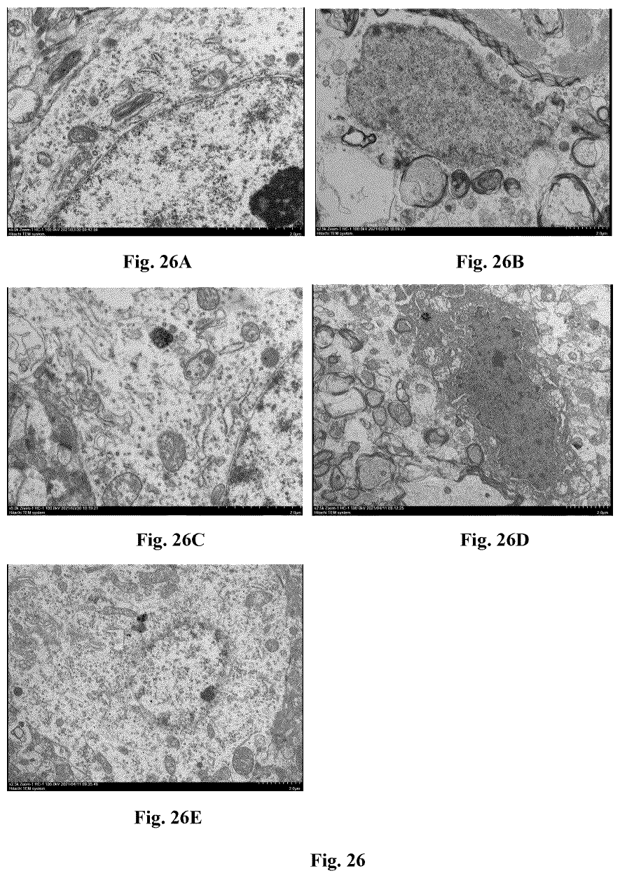

FIG. 26 shows the results of transmission electron microscope examination of ultra-thin sections of brain tissue.

As shown in FIG. 26A, in NC group, the morphology of glial cells was normal or slightly irregular; the nucleus (N) was irregularly oval; the mitochondria (M) in cytoplasm were short round or short rod-shaped, structural intact, slightly blurred inner ridge; and the endoplasmic reticulum (ER) was with slight expansion.

As shown in FIG. 26B, in SHAM group, the morphology of glial cells was slightly irregular; the nucleus (N) was oval; the mitochondria (M) in the cytoplasm were dumbbell shaped with blurred inner ridge; and the endoplasmic reticulum (ER) was slightly expanded.

As shown in FIG. 26C, in IVH group, the morphology of glial cells was irregular; the (N) was irregular; the mitochondria (M) in the cytoplasm were round and obviously swollen; the endoplasmic reticulum (ER) was obviously expanded; and autophagy bodies (AP) were visible; which shows that IVH modeling leads to serious damages of cells of brain tissue.

As shown in FIG. 26D, in A1 group, the morphology of glial cells was slightly irregular; the nucleus (N) was oval; the mitochondria (M) in the cytoplasm were round or oval, slightly swollen, some broken internal ridges; the endoplasmic reticulum (ER) was slightly expanded, and a small amount of lipofuscin (L) could be seen; which shows that low dose of drug A administration can improve or repair the cell damages of brain tissue caused by IVH modeling.

As shown in FIG. 26E, in AH group, the morphology of glial cells was basically regular or slightly irregular; the nucleus (N) was oval; the mitochondria (M) in cytoplasm were oval or long rod-shaped, with clear inner ridge and intact structure; the endoplasmic reticulum (ER) was slightly expanded; and a small amount of lipofuscin (L) could be seen in some parts; which shows that high dose of drug A administration can improve or repair the cell damages of brain tissue caused by IVH modeling, and that there is little difference of brain tissue cells between NC group and AH group.

The above results demonstrate that the gold cluster with L-NIBC as ligand has unexpected therapeutic effect on the treatment of hemorrhagic stroke, and can also be used in the development of therapeutic drugs for hemorrhagic stroke.

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the scope of the present invention. Accordingly, the scope of the present invention is defined by the appended claims and is supported by the foregoing description.

### REFERENCES

Amani H, Mostafavi E, Mahmoud Reza Alebouyeh M R, Arzaghi H, Akbarzadeh A, Pazoki-Toroudi H, Webster T J. Would Colloidal Gold Nanocarriers Present An Effective Diagnosis Or Treatment For Ischemic Stroke? Int J Nanomedicine. 2019 Oct. 7; 14:8013-8031.
Zheng Y, Wu Y, Liu Y, Guo Z, Bai T, Zhou P, Wu J, Yang Q, Liu Z, Lu X. Intrinsic Effects of Gold Nanoparticles on Oxygen-Glucose Deprivation/Reperfusion Injury in Rat Cortical Neurons. Neurochem Res. 2019 July; 44(7): 1549-1566.

1. A method for treating cerebral stroke in a subject, wherein the method comprises:
administering a composition to the subject with cerebral stroke;
wherein the composition comprises a ligand-bound gold cluster; and
a pharmaceutically acceptable excipient;
wherein the ligand-bound gold cluster comprises:
a gold core; and
a ligand bound to the gold core;
wherein the cerebral stroke is selected from the group consisting of cerebral hemorrhagic stroke, cerebral ischemic stroke, and transient ischemic attack (TIA).

2. The method of claim 1, wherein the gold core has a diameter in the range of 0.5-3 nm.

3. The method of claim 1, wherein the gold core has a diameter in the range of 0.5-2.6 nm.

4. The method of claim 1, wherein the ligand is one selected from the group consisting of L-cysteine and its derivatives, D-cysteine and its derivatives, cysteine-containing oligopeptides and their derivatives, and other thiol-containing compounds.

5. The method of claim 4, wherein the L-cysteine and its derivatives are selected from the group consisting of L-cysteine, N-isobutyryl-L-cysteine (L-NIBC), and N-acetyl-L-cysteine (L-NAC), and wherein the D-cysteine and its derivatives are selected from the group consisting of D-cysteine, N-isobutyryl-D-cysteine (D-NIBC), and N-acetyl-D-cysteine (D-NAC).

6. The method of claim 4, wherein the cysteine-containing oligopeptides and their derivatives are cysteine-containing dipeptides, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

7. The method of claim 4, wherein the cysteine-containing oligopeptides and their derivatives are cysteine-containing tripeptides, wherein the cysteine-containing tripeptides are selected from the group consisting of glycine-L(D)-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

8. The method of claim 4, wherein the cysteine-containing oligopeptides and their derivatives are cysteine-containing tetrapeptides, wherein the cysteine-containing tetrapeptides are selected from the group consisting of glycine-L(D)-serine-L(D)-cysteine-L(D)-arginine tetrapeptide (CSCR), and glycine-L(D)-cysteine-L(D)-serine-L(D)-arginine tetrapeptide (GCSR).

9. The method of claim 4, wherein the cysteine-containing oligopeptides and their derivatives are cysteine-containing pentapeptide, wherein the cysteine-containing pentapeptides are selected from the group consisting of Cysteine-Aspartic acid-Glutamic acid-Valine-Aspartic acid (CDEVD) and Aspartic acid-Glutamic acid-Valine-Aspartic acid-Cysteine (DEVDC).

10. The method of claim 4, wherein the other thiol-containing compounds are selected from the group consisting of 1-[(2S)-2-methyl-3-thiol-1-oxopropyl]-L(D)-proline, thioglycollic acid, mercaptoethanol, thiophenol, D-3-trolovol, N-(2-mercaptopropionyl)-glycine, dodecyl mercaptan, 2-aminoethanethiol (CSH), 3-mercaptopropionic acid (MPA), and 4-mercaptobenoic acid (p-MBA).

11. A pharmaceutical composition for treatment of cerebral stroke in a subject, wherein the pharmaceutical composition comprises a ligand-bound gold cluster; and a pharmaceutically acceptable excipient;
wherein the ligand-bound gold cluster comprises:
a gold core; and
a ligand bound to the gold core;
wherein the cerebral stroke is selected from the group consisting of cerebral hemorrhagic stroke, cerebral ischemic stroke, and transient ischemic attack (TIA).

12. The pharmaceutical composition of claim 11, wherein the gold core has a diameter in the range of 0.5-3 nm.

13. The pharmaceutical composition of claim 11, wherein the gold core has a diameter in the range of 0.5-2.6 nm.

14. The pharmaceutical composition of claim 11, wherein the ligand is one selected from the group consisting of L-cysteine and its derivatives, D-cysteine and its derivatives, cysteine-containing oligopeptides and their derivatives, and other thiol-containing compounds.

15. The pharmaceutical composition of claim 14, wherein the cysteine and its derivatives are selected from the group consisting of L-cysteine, N-isobutyryl-L-cysteine (L-NIBC), and N-acetyl-L-cysteine (L-NAC), and wherein the D-cysteine and its derivatives are selected from the group consisting of D-cysteine, N-isobutyryl-D-cysteine (D-NIBC), and N-acetyl-D-cysteine (D-NAC).

16. The pharmaceutical composition of claim 14, wherein the cysteine-containing oligopeptides and their derivatives are cysteine-containing dipeptides, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

17. The pharmaceutical composition of claim 14, wherein the cysteine-containing oligopeptides and their derivatives are cysteine-containing tripeptides, wherein the cysteine-containing tripeptides are selected from the group consisting of glycine-(D)L-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

18. The pharmaceutical composition of claim 14, wherein the cysteine-containing oligopeptides and their derivatives are cysteine-containing tetrapeptides, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

19. The pharmaceutical composition of claim 14, wherein the cysteine-containing oligopeptides and their derivatives are cysteine-containing pentapeptide, wherein the cysteine-containing pentapeptides are selected from the group consisting of Cysteine-Aspartic acid-Glutamic acid-Valine-Aspartic acid (CDEVD) and Aspartic acid-Glutamic acid-Valine-Aspartic acid-Cysteine (DEVDC).

20. The pharmaceutical composition of claim 14, wherein the other thiol-containing compounds are selected from the group consisting of 1-[(2S)-2-methyl-3-thiol-1-oxopropyl]-L(D)-proline, thioglycolic acid, mercaptoethanol, thiophenol, D-3-trolovol, N-(2-mercaptopropionyl)-glycine, dodecyl mercaptan, 2-aminoethanethiol (CSH), 3-mercaptopropionic acid (MPA), and 4-mercaptobenoic acid (p-MBA).
